(19) United States
(12) Patent Application Publication
(10) Pub. No.: US 2023/0109553 A1
(43) Pub. Date: Apr. 6, 2023

(54) CHIMERIC SCAVENGER RECEPTORS TARGETED TO PHOSPHORYLATED TAU (PTAU) AND USES THEREOF

(71) Applicant: Board of Regents, The University of Texas System, Austin, TX (US)

(72) Inventors: Michael A. CURRAN, Houston, TX (US); Chao-Hsien CHEN, Houston, TX (US)

(73) Assignee: Board of Regents, The University of Texas System, Austin, TX (US)

(21) Appl. No.: 17/904,473

(22) PCT Filed: Jan. 15, 2021
(86) PCT No.: PCT/US2021/013665

§ 371 Date: Aug. 17, 2022

Related U.S. Application Data
(60) Provisional application No. 63/069,896, filed on Aug. 25, 2020.
(60) Provisional application No. 62/977,626, filed on Feb. 17, 2020.

(51) Int. Cl. C07K 14/705; C07K 16/18; A61P 25/28

(52) U.S. Cl. CPC C07K 14/70596; C07K 16/18; A61P 25/28; A61K 2039/5156

(57) ABSTRACT

Provided herein are chimeric scavenger receptor where the endogenous binding region is replaced with an antigen-specific binding region to redirect the specificity of the receptor. Also provided are immune cells, such as, for example, monocytes, that are engineered to express the chimeric scavenger receptor. Also provided are methods of treating patients, such as, for example, Alzheimer's patients, by administering the engineered monocytes.

### REFERENCE TO RELATED APPLICATIONS

The present application claims the priority benefit of U.S. provisional application No. 63/069,896, filed Aug. 25, 2020 and U.S. provisional application No. 62/977,626, filed Feb. 17, 2020, the entire contents of each of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Dec. 9, 2020, is named UTFCP1502WO_ST25.txt and is 43.3 kilobytes in size.

### BACKGROUND

### 1. Field

The present invention relates generally to the fields of medicine and immunology. More particularly, it concerns compositions and methods for stimulating antigen-targeted anti-inflammatory responses. Even more particularly, it concerns monocyte-based cellular therapy using chimeric scavenger receptors for the treatment of neurodegenerative diseases.

### 2. Description of Related Art

The health challenges posed by Alzheimer's disease (AD) continue to grow as societies age worldwide. Accumulation of hyperphosphorylated Tau (pTau) and its associated pathology correlates with clinical cognitive deterioration in AD. Resident myeloid cells (i.e., microglia) within the central nervous system (CNS) have a limited capacity to uptake and degrade pTau; however, the resulting secretion of proinflammatory cytokines only acts to accelerate neurodegeneration. Therapeutic antibodies can reduce the neurotoxic oligomeric form of pTau (o-pTau), but in doing so they also aggravate inflammation. These processes may also be related to cerebral hemorrhage observed in some antibody-treated AD patients. Attenuating mutation of the antibody Fc region can silence inflammation but also eliminates its capacity to mediate o-pTau clearance by central nervous system (CNS) myeloid cells. Thus, there is an unmet need for a therapeutic that catalyzes o-pTau degradation without triggering neurotoxic inflammation.

### SUMMARY

Provided herein are compositions and methods for the treatment of neurodegenerative disease, namely a monocyte based cellular therapy which relies on chimeric scavenger receptors to direct clearance of Tau from the brain without neurotoxic levels of cytokine release.

In one embodiment, provided herein are chimeric scavenger receptors (CSRs) comprising (1) an intracellular signaling domain, wherein the intracellular domain comprises an intracellular domain of a scavenger receptor; (2) a transmembrane domain; and (3) an extracellular domain, wherein the extracellular domain comprises an antigen-specific binding domain. In some aspects, the transmembrane domain comprises a transmembrane domain of a scavenger receptor. In some aspects, the scavenger receptor is FcγRIIb, CD163, CD204, or CD206. In some aspects, the extracellular domain comprises an extracellular scaffold of a scavenger receptor with the antigen-specific binding domain grafted in place of the scavenger receptor's endogenous binding domain. In some aspects, the antigen-specific binding domain comprises an scFv sequence. In some aspects, the antigen-specific binding domain comprises a single chain monoclonal antibody to any form of the Tau protein. In some aspects, the single chain monoclonal antibody to o-pTau comprises a variable light chain having a sequence at least 95% identical to

DIVMTQTPLSLPVTPGEPASISCRSSQSLVHSHGRTYLHWYLQKPGQSPQLLIYKVSN RFFGVPDRFSGSGSGTDFTLKISRVEAEDVGVYFCSQTRHFPYTFGGGTKVEIK (SEQ ID NO: 6). In some aspects, the single chain monoclonal antibody to o-pTau comprises a variable heavy chain having a sequence at least 95% identical to

(SEQ ID NO: 7)
EVQLVQSGAEVKKPGSSVKVSCKASGYTFTDYYMNWVRQAPGQGLEWIG
DINPNRGGTTYNQKFKGRVTITVDKSTSTAYMELSSLRSEDTAVYYCAS
YYAVGYWGQGTTVTVSS.

In one embodiment, provided herein are chimeric scavenger receptors (CSRs) comprising (1) an intracellular signaling domain, wherein the intracellular domain comprises an intracellular domain of a scavenger receptor; (2) a transmembrane domain; and (3) an extracellular domain, wherein the extracellular domain comprises an antigen-specific binding domain, wherein the antigen-specific binding domain comprises an scFv sequence, wherein the scFv sequence has a variable light chain having a sequence at least 95% identical to

DIVMTQTPLSLPVTPGEPASISCRSSQSLVHSHGRTYLHWYLQKPGQSPQLLIYKVSN RFFGVPDRFSGSGSGTDFTLKISRVEAEDVGVYFCSQTRHFPYTFGGGTKVEIK (SEQ ID NO: 6), and wherein the scFv sequence has a heavy chain having a sequence at least 95% identical to
EVQLVQSGAEVKKPGSSVKVSCKASGYTFTDYYMNWVRQAPGQGLEWIGDINPNR GGTTYNQKFKGRVTITVDKSTSTAYMELSSLRSEDTAVYYCASYYAVGYWGQGTT VTVSS (SEQ ID NO: 7). In some aspects, the transmembrane domain comprises a transmembrane domain of a scavenger receptor. In some aspects, the scavenger receptor is FcγRIIb, CD163, CD204, or CD206.

In one embodiment, provided herein are chimeric scavenger receptors (CSRs) comprising (1) an intracellular signaling domain, wherein the intracellular domain comprises an intracellular domain of a scavenger receptor, wherein the scavenger receptor is CD163, CD204, or CD206; (2) a transmembrane domain; and (3) an extracellular domain, wherein the extracellular domain comprises an antigen-specific binding domain. In some aspects, the transmembrane domain comprises a transmembrane domain of the scavenger receptor. In some aspects, the extracellular domain comprises an extracellular scaffold of a scavenger receptor with the antigen-specific binding domain grafted in place of the scavenger receptor's endogenous binding domain. In some aspects, the antigen-specific binding domain comprises an scFv sequence. In some aspects, the antigen-specific binding domain comprises a single chain monoclonal antibody to any form of the Tau protein. In some aspects, the single chain monoclonal antibody to o-pTau comprises a variable light chain having a sequence at least 95% identical to

DIVMTQTPLSLPVTPGEPASISCRSSQSLVHSHGRTYLHWYLQKPGQSPQLLIYKVSN RFFGVPDRFSGSGSGTDFTLKISRVEAEDVGVYFCSQTRHFPYTFGGGTKVEIK (SEQ ID NO: 6). In some aspects, the single chain monoclonal antibody to o-pTau comprises a variable heavy chain having a sequence at least 95% identical to

(SEQ ID NO: 7)
EVQLVQSGAEVKKPGSSVKVSCKASGYTFTDYYMNWVRQAPGQGLEWIG
DINPNRGGTTYNQKFKGRVTITVDKSTSTAYMELSSLRSEDTAVYYCAS
YYAVGYWGQGTTVTVSS.

In some aspects of the any of the above embodiments, the cytoplasmic tail is replaced by the cytoplasmic tail of an anti-inflammatory cytokine receptor. In some aspects of the any of the above embodiments, the receptor is an IL-4 receptor, IL-10 receptor, IL-11 receptor, IL-13 receptor, IL-27 receptor, IL-33 receptor, IL-35 receptor, TGF-β receptor, or TSLP receptor.

In some aspects of the any of the above embodiments, the variable light chain and variable heavy chain are separated by a linker sequence. In some aspects, the linker sequence is GGGGSGGGGSGGGGS (SEQ ID NO: 8).

In some aspects of the any of the above embodiments, the CSRs further comprise a hinge sequence positioned between the transmembrane domain and the antigen-specific binding domain. In some aspects, the hinge sequence is VPRDCGCKPCICTV (SEQ ID NO: 9).

In some aspects of the any of the above embodiments, the chimeric scavenger receptors comprise a sequence at least 95% identical to the sequence of SEQ ID NO: 1, 5, or 11. In some aspects of the any of the above embodiments, the chimeric scavenger receptors comprise a sequence at least 95% identical to the sequence of SEQ ID NO: 2 or 4. In some aspects of the any of the above embodiments, the chimeric scavenger receptors comprise a sequence at least 95% identical to the sequence of SEQ ID NO: 3.

In some aspects of the any of the above embodiments, the chimeric scavenger receptors comprise the sequence of SEQ ID NO: 1, 5, or 11. In some aspects of the any of the above embodiments, the chimeric scavenger receptors comprise the sequence of SEQ ID NO: 2 or 4. In some aspects of the any of the above embodiments, the chimeric scavenger receptors comprise the sequence of SEQ ID NO: 3.

In one embodiment, provided herein are nucleic acids encoding a CSR of any one of the present embodiments. In some aspects, the sequence encoding the CSR is operatively linked to an expression control sequence.

In one embodiment, provided herein are viral vectors comprising the nucleic acid encoding the CSR of any one of the present embodiments. In some aspects, the viral vector is a retroviral vector. In some aspects, the viral vector is a lentiviral vector. In some aspects, the viral vectors further comprise a drug selection marker.

In one embodiment, provided herein are engineered immune effector cells comprising the nucleic acid of any one of the present embodiments. In some aspects, the immune effector cell is an immune suppressive myeloid cell. In some aspects, the immune effector cell is a CD11b and/or CD11c positive myeloid cell. In some aspects, the immune effector cell is a macrophage. In some aspects, the immune effector cell is a monocyte. In some aspects, the engineered immune effector cell secretes lower levels of pro-inflammatory cytokines than an equivalent parental immune effector cell. In some aspects, the pro-inflammatory cytokine is TNF-α or IL-6. In some aspects, the immune effector cell is a human immune effector cell.

In one embodiment, provided herein are pharmaceutical formulations comprising the engineered cell of any one of the present embodiments and a pharmaceutically acceptable carrier.

In one embodiment, provided herein are methods of treating Alzheimer's disease, frontotemporal dementia, a tauopathy, or a Tau protein-associated impairment in or loss of cognitive function in a patient in need thereof, the method comprising administering an effective amount of a cellular immunotherapy to the patient, wherein the cellular immunotherapy targets a Tau protein. In some aspects, the Tau protein is pTau. In some aspects, the Tau protein is o-pTau.

In some aspects, the methods reduce the levels of Tau, pTau, and/or o-pTau in the brain of the patient. In some aspects, the methods prevent cognitive deterioration in the patient. In some aspects, the methods improve cognitive function in the patient. In some aspects, the methods prevent neural damage in the patient. In some aspects, the methods prevent neuronal fragmentation in the patient.

In some aspects, the cellular immunotherapy is administered to the patient using intraventricular injection. In some aspects, the cellular immunotherapy is administered to the patient using an intracerebroventricular reservoir. In some aspects, the cellular immunotherapy is administered by intrathecal injection.

In some aspects, the cellular immunotherapy comprises an engineered cell of any one of the present embodiments. In some aspects, the engineered cell is autologous to the patient. In some aspects, the patient is a human.

In one embodiment, provided herein are engineered cells of any one of the present embodiments, for use as a medicament.

In one embodiment, provided herein are engineered cells of any one of the present embodiments, for use in treating Alzheimer's disease, frontotemporal dementia, a tauopathy, or a Tau protein-associated impairment in or loss of cognitive function in a patient.

In one embodiment, provided herein is the use of the engineered cells of any one of the present embodiments, in the manufacture of a medicament for treating Alzheimer's disease, frontotemporal dementia, a tauopathy, or a Tau protein-associated impairment in or loss of cognitive function in a patient.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. The o-pTau CSR design. The o-pTau specific ScFv including a pair of heavy (VH) and light chain (VL) of immunoglobulin variable region is linked to scavenger receptor scaffold with an intact intracellular signaling domain.

FIGS. 2A-2C. CSR-monocytes increase o-pTau uptake with reduced inflammatory cytokine release. After 24 hours of incubation with o-pTau, CSR-monocytes secrete reduced proinflammatory cytokines including TNF-α (FIG. 2A) and IL-6 (FIG. 2B) and have higher cell-associated o-pTau (FIG. 2C) (n=1-5).

FIG. 3. Co-localization of internalized o-pTau with lysosome in FcγRIIb-CSR-monocytes. Amnis® imaging of CSR-monocytes after 6-hour incubation with fluorescently labeled o-pTau (green) and lysosome is stained with lysosome associated membrane protein-1 (LAMP-1) (red).

FIGS. 4A-4B FcγRIIb-CSR-monocytes limit neuronal damage in vitro. Primary E18 neurons (200,000 neurons/well) were incubated for 72 h with 500 nM o-pTau and either parental or CSR-engineered monocytes (500,000 monocytes/well). Compared to multiple fragmentations detected by MAP2 antibody on the primary hippocampal neurons co-cultured with parental monocytes (FIG. 4A, arrow heads), neurons co-cultured with CSR-monocytes have reduced fragmentation on their processes (FIG. 4B).

FIG. 5. Generation and purification of oligomeric pTau (o-pTau) utilizing size exclusion chromatography.

FIGS. 6A-6B. FcγRIIb-CSR-monocytes have enhanced cell-associated o-pTau. After incubation with o-pTau at either 0 nM or 500 nM for 24 hours, the monocytes were lysed for detection of cell-associated o-pTau using Western blot (FIG. 6A). Quantification of cell-associated o-pTau versus actin ratio at 500 nM incubation shows enhanced o-pTau association in CSR-monocytes (n=4) (FIG. 6B).

FIGS. 7A-7B. FcγRIIb-CSR-monocytes have a trend of o-pTau reduction in the supernatant. Remaining o-pTau in supernatant after 24-hour incubation with parental or CSR monocytes was determined by Western blot (n=1) (FIG. 7A) and ELISA (n=1) (FIG. 7B).

FIG. 8. CSR-monocytes have a higher lysosomal consumption activity post o-pTau stimulation. LAMP-1 staining indicates lysosomal consumption activity (Yu et al., 2010) at different time points post o-pTau stimulation using flow cytometry. LAMP-1 fold change is relative to the mean fluorescence intensity (MFI) at 0-hr in the respective groups.

FIG. 9. Fragmentation of MAP2 in primary neurons is prevented by co-culture with CSR-monocytes. Primary hippocampal neurons (15,000 neurons/well) co-cultured with o-pTau and monocytes (30,000 or 60,000 monocytes/well) are stained for MAP2 (white). Significant MAP2 fragmentation (arrow heads) is observed post o-pTau, and even worse in co-culture with control monocytes. But the fragmentation is prevented by CSR-monocytes, even at a higher number of monocytes.

FIG. 10. Engineered monocytes secrete less pro-inflammatory cytokines. After 24 hours of incubation with 500 nM o-pTau, CSR-monocytes secrete reduced proinflammatory cytokines including TNF-α and IL-6 than parental monocytes (N=5, n=3). In each pair of columns, the left column represent Parental monocytes and the right column represents Engineered monocytes.

FIG. 11. Engineered monocytes secrete less pro-inflammatory cytokines. After 72 hours of incubation with 500 nM o-pTau, either with or without parental or CSR-engineered monocytes, CSR-monocytes secrete reduced proinflammatory cytokines including TNF-α and IL-6 than parental monocytes.

FIG. 12. Cytokine secretion by myeloid cells in response to pTau. Peripheral monocytes and a monocyte cell line (PMJ2-PC), similar to a microglia cell lines (BV2) secrete the pro-inflammatory cytokine, TNFα, upon pTau stimulation.

FIG. 13. Cell-associated o-pTau on FcγIIb-CSR-monocytes is intracellular. Cells were incubated with o-pTau (125 nM) for the indicated length of time.

FIG. 14. O-pTau ScFv-FcγRIIb CSR construct schematic (SEQ ID NO: 1).

FIG. 15. O-pTau ScFv-CD163 CSR construct schematic (SEQ ID NO: 2).

FIG. 16. O-pTau ScFv-CD204 CSR construct schematic (SEQ ID NO: 3).

FIG. 17. O-pTau ScFv-modified CD163 CSR construct schematic (SEQ ID NO: 4).

FIG. 18. O-pTau ScFv-modified FcγRIIb2 CSR construct schematic (SEQ ID NO: 5). The FcγIIb1 specific sequence is provided as SEQ ID NO: 10, and the O-pTau ScFv-modified FcγRIIb1 CSR construct is provided as SEQ ID NO: 11.

FIGS. 19A-19D. Association of o-pTau uptake and proinflammatory cytokine secretion by the engineered monocytes. Parental and CSR-monocyte cell lines were incubated with 0, 125, or 500 nM of o-pTau for 24 hours before evaluation of their cell-associated o-Tau by using Western blot (FIG. 19A), and proinflammatory cytokine secretion by using Cytometric Bead Assays (FIG. 19B). The correlation between cytokine secretion with o-pTau stimulation (FIG. 19C) or with o-pTau uptake (FIG. 19D) was determined by Pearson correlation analyses and the association was demonstrated by linear regression analyses (FIG. 19C) (N=1, n=3). Unpaired Student's t-test in FIG. 19B, ns=not significant, *=p<0.05, **=p<0.01, ****=p<0.0001.

FIGS. 20A-20F. Co-culture of primary neurons with various doses of the FcγRIIb-CSR monocytes. Graphic representation of TNF secretion from monocytes in co-culture (FIG. 20A). Representative images show primary neurons in various co-culture conditions (FIG. 20B-F). 15×10³ E18 primary neurons were cultured in vitro for 14 days before co-culture with 30×10³ parental monocytes, 30×10³ or 60×10³ FcγRIIb-CSR monocytes in addition to 500 nM of o-pTau. After 72 hours of incubation, the neurons were fixed and stained with MAP2 antibody, then imaged using the Operetta high-content imager.

FIGS. 21A-21D. Motor tests of the P301S mice treated with engineered monocytes. Male P301S mice received eight doses of weekly infusion of either PBS vehicle, control monocytes, or FcγRIIb-CSR monocytes since they were 4 months old. Motor tests including wire hang test (FIG. 21A) and footprint tests (FIGS. 21B-D) were performed at the end of eight treatments when the mice were 6 months old. p-values were calculated using unpaired Student's t test.

FIGS. 22A-22C. Monocytes transduced with the FcγRIIb1-CSR, FcγRIIb2-CSR, or control vector were incubated with 500 nM of o-Tau at 37° C. for 24 hours. TNF secretion from the monocytes was quantified using ELISA (FIGS. 22A and B), and cell-associated o-Tau of the monocytes was determined by Western Blot (FIG. 22C).

### DETAILED DESCRIPTION

Provided herein are chimeric scavenger receptors, such as, for example, FcγRIIb, CD163, CD204, and CD206, that have been repurposed to facilitate o-pTau internalization in AD while limiting inflammation. The o-pTau specific ScFv used in the CSR constructs provided herein not only serves to enhance neurotoxic o-pTau clearance, but also redirects monocyte responses by signaling through anti-inflammatory scavenger receptors that are specialized at mediating internalization and degradation of their substrates without triggering an inflammatory burst. This design not only attenuates Tau pathology, but also overcomes the concomitant inflammation, an overarching challenge in the AD field. These CSRs may be transduced into monocytes that may be introduced into the brain of patients suffering from neurodegeneration in order to clear neurotoxic Tau and slow or reverse disease progression. Unlike microglia in late stage AD, monocytes retain the ability to clear pTau assemblies, but also secrete inflammatory cytokines.

A peripheral macrophage/monocyte cell line was successfully engineered to stably express the CSR consisting of a scavenger receptor scaffold and an anti-o-pTau single-chain variable fragment. These engineered CSR-monocytes not only mediate enhanced removal of extracellular o-pTau, but also uncouple phagocytosis from neurotoxic proinflammatory cytokine production. The majority of the monocyte-associated o-pTau is internalized where it co-localizes with lysosomes, likely as a prelude to proteolytic degradation. CSR monocytes demonstrated an elevated lysosome consumption activity relative to parental monocytes, indicative of a similarly higher degradative capacity. In primary culture, CSR-monocytes protected primary neurons from o-pTau-induced cytopathology. To validate the therapeutic efficacy of these CSR monocytes in relevant in vivo preclinical models, a minimally invasive procedure was established for repeated intracerebroventricular adoptive transfer of these cellular therapeutics. This is the first proof of concept that myeloid cell-based immunotherapies engineered to safely target and reduce Tau pathology through CSR expression can be harnessed to treat neurodegenerative diseases such as AD. Monocytes engineered to bind and internalize o-pTau via antibody-redirected chimeric scavenger receptors (CSR) halted the progression of AD by protecting neurons from o-pTau-mediated neurotoxicity while dampening proinflammatory cytokine release.

Such chimeric scavenger receptors (such as, for example, FcγRIIb, CD163, CD204, CD206) may be engineered to target any specific antigen by replacing the antigen binding domain with an scFv sequence that targets the antigen. Then, patient monocytes can be engineered using viral vectors to express the chimeric scavenger receptor. These monocytes may then be administered to the patient in order to target, internalize, and degrade the specific antigen which is causing their disease to progress.

### I. Tauopathies

Aspects of the present invention relate to “tauopathies.” As well as Alzheimer's disease (AD), the pathogenesis of neurodegenerative disorders such as Pick's disease and Progressive Supranuclear Palsy (PSP) appears to correlate with an accumulation of pathological truncated tau aggregates in the dentate gyms and stellate pyramidal cells of the neocortex, respectively. Other dementias include frontotemporal dementia (FTD); parkinsonism linked to chromosome 17 (FTDP-17); disinhibition-dementia-parkinsonism-amyotrophy complex (DDPAC); pallido-ponto-nigral degeneration (PPND); Guam-ALS syndrome; pallido-nigro-luysian degeneration (PNLD); cortico-basal degeneration (CBD) and others. All of these diseases, which are characterized primarily or partially by abnormal tau aggregation, are referred to herein as “tauopathies” or “diseases of tau protein aggregation.”

Alzheimer's disease (AD) is a central nervous system (CNS) neurodegenerative disease characterized by progressive memory loss, cognitive disturbances, and functional decline leading to loss of quality of life and long-term dependency. As the population ages, AD is projected to affect more than 130 million people worldwide by 2050 (Hebert et al., 2013). Despite this massive burden to society, there is no disease-modifying treatment available yet. Development of effective treatment for AD has been exceptionally challenging due in part to its multifactorial and intertwined pathogenesis, such as self-propagating protein pathologies and inflammation (Spangenberg & Green, 2017; Heppner et al., 2015). One critical pathognomonic feature of AD is the accumulation of intracellular hyperphosphorylated Tau protein (pTau) tangles in the CNS which, unlike β-amyloid (Aβ) plaques, are closely associated with neuronal loss and clinical dementia progression in AD patients (Karran et al., 2011; Nelson et al., 2009; Braak et al., 2016).

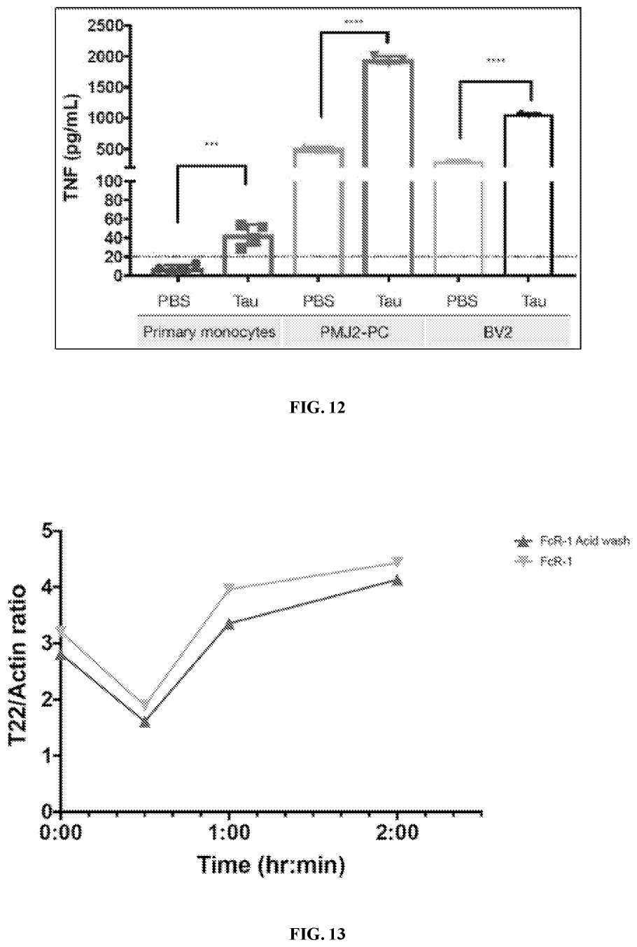

More than a disease of proteinopathy, AD is also a disease of chronic inflammation (Heppner et al., 2015). Microglia are the major innate immune cells in the CNS responsible for the phagocytosis and degradation of waste products, including pTau assemblies (Bolos et al., 2016). As AD progresses, microglia lose their capability for pTau clearance but secrete more proinflammatory cytokines, such as tumor necrosis factor (TNF) α, interleukin (IL)-1β and IL-6 (Lee et al., 2016; Bolos et al., 2017). Although peripheral monocytes recruited into the CNS retain the ability to clear pTau assemblies (Fiala et al., 1998; Lebson et al., 2010; Majerova et al., 2019; Majerova et al., 2014), they also release proinflammatory cytokines in response (FIG. 12). The reactive microglia and the recruited monocytes along with their secreted proinflammatory cytokines in turn accelerate the progression of Tau pathology and cognitive decline (Maphis et al., 2015; Neniskyte et al., 2014; Ghosh et al., 2013; Kitazawa et al., 2011; Li et al., 1997; Bellinger et al., 1995). Breaking this vicious cycle between these two pathologies provides a novel approach to halt AD progression by halting the accumulation of pTau and limiting neuroinflammation. However, previous efforts to curb inflammation by systemic administration of non-steroid anti-inflammatory drugs (NSAIDs) have failed to protect cognitive decline in high-risk AD populations, which highlight the complex differential effects of cytokines in AD (Group, 2015; Meyer et al., 2019).

Oligomeric pTau (o-pTau), but not monomer nor tangles, has been shown to be deleterious to cognitive function (Lasagna-Reeves et al., 2011). Accumulation of o-pTau can be restricted by administration of monoclonal antibodies specific for o-pTau. However, this approach instigates inflammatory responses by CNS myeloid cells that lead to neurotoxic cytokine secretion and intensify neuronal pTau production (Lee et al., 2016). These processes may also be related to adverse events including vasogenic edema and microhemorrhages in the CNS observed in some antibody-treated AD patients, which limit the use and efficacy of this antibody (Vandenberghe et al., 2016; Sperling et al., 2011). Proinflammatory cytokine production can be prevented by modifying the Fc effector region; however, this modification also impairs o-pTau clearance by CNS myeloid cells (Lee et al., 2016).

Previous efforts focusing on single etiology such as targeting Aβ plaques with monoclonal antibodies and/or vaccines or curbing inflammation by NSAIDs failed to show clinical benefit (Group, 2015; Meyer et al., 2019; Panza et al., 2019). Trials of monoclonal antibodies against the more clinically relevant Tau pathology have yet to report positive findings in the clinic (Medina, 2018). Moreover, the antibody approach has been shown to induce undesired inflammatory responses. Modification of the Fc portion of the monoclonal antibody to prevent inflammation, in turn, impairs the clearance of pTau (Lee et al., 2016).

The present invention provides compositions and methods for the simultaneous targeting, with a single therapeutic, of both the buildup of o-pTau and the concomitant neurotoxic inflammation that results.

The term “Tau,” as used herein, refers to any native Tau protein from any vertebrate source, including mammals such as primates (e.g., humans) and rodents (e.g., mice and rats), unless otherwise indicated. The term encompasses “full-length,” unprocessed Tau as well as any form of Tau that results from processing in the cell. The term also encompasses naturally occurring variants of Tau, e.g., splice variants or allelic variants.

The term “pTau,” as used herein, refers to Tau in which a serine, a threonine or a tyrosine residue is phosphorylated by a protein kinase by the addition of a covalently bound phosphate group. In some embodiments, pTau is phosphorylated on a serine or on a threonine residue. In some embodiments, pTau is phosphorylated on Serine at position 409 and/or Serine at position 404. In one embodiment, pTau is phosphorylated on Serine at position 409.

The term “oligomeric Tau,” as used herein, refers to multiple aggregated monomers of Tau peptides or proteins, or of Tau-like peptides/proteins, or of modified or truncated Tau peptides/proteins or of other derivates of Tau peptides/proteins forming oligomeric or polymeric structures which are insoluble or soluble both in vitro in aqueous medium and in vivo in the mammalian or human body more particularly in the brain, but particularly to multiple aggregated monomers of Tau or of modified or truncated Tau peptides/proteins or of derivatives thereof, which are insoluble or soluble in the mammalian or human body more particularly in the brain, respectively.

### II. Engineered Myeloid Cells

In certain embodiments, the present disclosure provides methods for producing chimeric scavenger receptor engineered myeloid cells comprising transducing the cells with a CSR construct. The CSR construct may be a retroviral or lentiviral vector, utilize transposon-mediated gene transfer, or may be electroporated. A myeloid cell is any CD11b and/or CD11c positive myeloid cells, such as, for example, a macrophage, a microglial cell, or a monocyte.

A “monocyte cell” is a large mononuclear phagocyte of the peripheral blood. Monocytes vary considerably, ranging in size from 10 to 30 μm in diameter. The nucleus to cytoplasm ratio ranges from 2:1 to 1:1. The nucleus is often band shaped (horseshoe), or reniform (kindey-shaped). It may fold over on top of itself, thus showing brainlike convolutions. No nucleoli are visible. The chromatin pattern is fine and arranged in skein-like strands. The cytoplasm is abundant and appears blue gray with many fine azurophilic granules, giving a ground glass appearance in Giemsa staining Vacuoles may be present. More preferably, the expression of specific surface antigens is used to determine whether a cell is a monocyte cell. The main phenotypic markers of human monocyte cells include CD11b, CD11c, CD33 and CD115. Generally, human monocyte cells express CD9, CD11b, CD11c, CDw12, CD13, CD15, CDw17, CD31, CD32, CD33, CD35, CD36, CD38, CD43, CD49b, CD49e, CD49f, CD63, CD64, CD65s, CD68, CD84, CD85, CD86, CD87, CD89, CD91, CDw92, CD93, CD98, CD101, CD102, CD111, CD112, CD115, CD116, CD119, CDw121b, CDw123, CD127, CDw128, CDw131, CD147, CD155, CD156a, CD157, CD162, CD163, CD164, CD168, CD171, CD172a, CD180, CD206, CD131a1, CD213a2, CDw210, CD226, CD281, CD282, CD284, CD286 and optionally CD4, CD14, CD16, CD40, CD45RO, CD45RA, CD45RB, CD62L, CD74, CD142 and CD170, CD181, CD182, CD184, CD191, CD192, CD194, CD195, CD197, CX3CR1. The main phenotypic markers of mouse monocyte cells include CD11b+, CD115, F4/80+. Generally, mouse monocytes express CD11a, CD11b, CD16, CD18, CD29, CD31, CD32, CD44, CD45, CD49d, CD115, CD116, Cdw131, CD281, CD282, CD284, CD286, F4/80, and optionally CD49b, CD62L, CCR2, CX3CR1, and Ly6C. Upon contact with sensitive target cells, monocyte cells also produce a number of cytokines, including IFNs, TNFs, GM-CSF, G-CSF, M-CSF, and IL-1.

The monocytes may be expanded in a functionally closed system, such as a bioreactor. Expansion may be performed in a gas-permeable bioreactor, such as G-Rex cell culture device. The bioreactor may support between 1×109 and 3×109 total cells in an average 450 mL volume. Bioreactors can be grouped according to general categories including: static bioreactors, stirred flask bioreactors, rotating wall vessel bioreactors, hollow fiber bioreactors and direct perfusion bioreactors. Within the bioreactors, cells can be free, or immobilized, seeded on porous 3-dimensional scaffolds (hydrogel).

The cells may be seeded in the bioreactor at a density of about 100-1,000 cells/cm2, such as about 150 cells/cm2, about 200 cells/cm2, about 250 cells/cm2, about 300 cells/cm2, such as about 350 cells/cm2, such as about 400 cells/cm2, such as about 450 cells/cm2, such as about 500 cells/cm2, such as about 550 cells/cm2, such as about 600 cells/cm2, such as about 650 cells/cm2, such as about 700 cells/cm2, such as about 750 cells/cm2, such as about 800 cells/cm², such as about 850 cells/cm², such as about 900 cells/cm², such as about 950 cells/cm², or about 1000 cells/cm². Particularly, the cells may be seeded at a cell density of about 400-500 cells/cm², such as about 450 cells/cm².

The total number of cells seeded in the bioreactor may be about 1.0×10⁶ to about 1.0×10⁸ cells, such as about 1.0×10⁶ to 5.0×10⁶, 5.0×10⁶ to 1.0×10⁷, 1.0×10⁷ to 5.0×10⁷, 5.0×10⁷ to 1.0×10⁸ cells. In particular aspects, the total number of cells seeded in the bioreactor are about 1.0×10⁷ to about 3.0×107, such as about 2.0×107 cells.

The cells may be seeded in any suitable cell culture media, many of which are commercially available. Exemplary media include DMEM, RPMI, MEM, Media 199, HAMS and the like. In one embodiment, the media is alpha MEM media, particularly alpha MEM supplemented with L-glutamine. The media may be supplemented with one or more of the following: growth factors, cytokines, hormones, or B27, antibiotics, vitamins and/or small molecule drugs. The media may be serum-free.

In some embodiments the cells may be incubated at room temperature. The incubator may be humidified and have an atmosphere that is about 5% CO₂ and about 1% O₂. In some embodiments, the CO₂ concentration may range from about 1-20%, 2-10%, or 3-5%. In some embodiments, the O₂ concentration may range from about 1-20%, 2-10%, or 3-5%.

The monocytes of the present disclosure can be genetically engineered to express chimeric scavenger receptors having antigenic specificity for a target antigen. For example, the monocyte cells are modified to express a CSR having antigenic specificity for a Tau protein.

Suitable methods of modification are known in the art. In some embodiments, the monocytes comprise one or more nucleic acids introduced via genetic engineering that encode one or more CSR, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CSR contains an extracellular antigen-recognition domain that specifically binds to an antigen. In some embodiments, the antigen is a protein expressed on the surface of cells or present in extracellular space. In some embodiments, the CSR comprises: a) an intracellular signaling domain, b) a transmembrane domain, and c) an extracellular domain comprising an antigen binding region. In certain embodiments, the antigen binding region can comprise complementarity determining regions of a monoclonal antibody, variable regions of a monoclonal antibody, and/or antigen binding fragments thereof. In another embodiment, that specificity is derived from a peptide (e.g., cytokine) that binds to a receptor. The antigen binding region can comprise a fragment of the V_{H} and V_{L} chains of a single-chain variable fragment (scFv) derived from a particular monoclonal antibody, such as those described in U.S. Pat. No. 9,598,485, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CSR includes a transmembrane domain fused to the extracellular domain of the CSR. In one embodiment, the transmembrane domain that naturally is associated with one of the domains in the CSR is used. In some instances, the transmembrane domain is selected or modified by amino acid substitution to avoid binding of such domains to the transmembrane domains of the same or different surface membrane proteins to minimize interactions with other members of the receptor complex.

The CSR of the immune cells of the present disclosure may comprise one or more suicide genes. The term “suicide gene” as used herein is defined as a gene which, upon administration of a prodrug, effects transition of a gene product to a compound which kills its host cell. Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include CD20, CD52, EGFRv3, or inducible caspase 9. In one embodiment, a truncated version of EGFR variant III (EGFRv3) may be used as a suicide antigen which can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

One of skill in the art would be well-equipped to construct a vector through standard recombinant techniques (see, for example, Sambrook et al., 2001 and Ausubel et al., 1996, both incorporated herein by reference) for the expression of the CSRs of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also contemplates degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression.

An “enhancer” is a DNA sequence that can stimulate transcription activity and may be an innate element of the promoter or a heterologous element that enhances the level or tissue specificity of expression. It is capable of operating in either orientation (5′−>3′ or 3′−>5′), and may be capable of functioning even when positioned either upstream or downstream of the promoter.

Promoters and/or enhancers may be derived in their entirety from a native gene, or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein, and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the invention, such polypeptides encoded by nucleic acid sequences are not required to be identical to the endogenous protein that is defective, or whose activity, function, or expression is insufficient, deficient or absent in a treated mammal

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues).

An example of an amino acid modification is a conservative amino acid substitution or a deletion. In particular embodiments, a modified or variant sequence retains at least part of a function or activity of the unmodified sequence (e.g., wild-type sequence).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

### III. Pharmaceutical Formulations

In some embodiments, the composition comprises at least 2×10⁶ monocytes expressing a CSR, at least 1×10⁶ monocytes expressing a CSR, at least 5×10⁶ monocytes expressing a CSR, or at least 10×10⁶ monocytes expressing a CSR. In some embodiments, the disclosed methods comprised repeated administrations of the compositions, for instance repeating administration of the composition at least five times, repeating administration of the composition at least ten times, or repeating administration for the life of the patient. In some embodiments, the administration is repeated once a week or once every two weeks. In some embodiments, the monocytes are autologous to the patient.

In some embodiments, an effective amount of monocytes expressing a CSR ranges from about 1×10⁶ monocytes to about 100×10⁶ monocytes, and in some embodiments, an effective amount of monocytes ranges from about 2×10⁶ monocytes to about 50×10⁶ monocytes.

Also disclosed are methods comprising intraventricular administration of a population of human monocytes transduced by a vector comprising an expression cassette encoding a chimeric scavenger receptor, wherein chimeric scavenger receptor comprises: a transmembrane domain selected from: a CD163 transmembrane domain or variant thereof having 1-10 amino acid modifications, a CD204 transmembrane domain or variant thereof having 1-10 amino acid modifications, a CD206 transmembrane domain or a variant thereof having 1-10 amino acid modifications, and a FcγRIIb transmembrane domain or a variant thereof having 1-10 amino acid modifications; and an intracellular signaling domain selected from: CD163 intracellular signaling domain (either with SCRC3-9 or with SCRC3-4) or variant thereof having 1-10 amino acid modifications, a CD204 intracellular signaling domain or variant thereof having 1-10 amino acid modifications, a CD206 intracellular signaling domain or a variant thereof having 1-10 amino acid modifications, and a FcγRIIb (either FcγRIIb1 or FcγRIIb2) intracellular signaling domain or a variant thereof having 1-10 amino acid modifications. In various embodiments: the population of human monocytes comprise a vector expressing a chimeric scavenger receptor comprising an amino acid sequence selected from SEQ ID NOs: 1-5.

In some embodiments the monocytes administered to the patient harbor a nucleic acid molecule that expresses a polypeptide comprising an amino acid sequence selected from SEQ ID NOs: 1-5. The disclosed methods of treatment using CSR monocytes can be performed at various doses and across various timeframes. For example, a patient receiving an infusion, administration, or injection of CSR monocytes may receive a single dose comprising between 1×10⁶ and 15×10⁶ cells. In other words, a single dose for use in the disclosed methods can comprise 1×10⁶, 2×10⁶, 3×10⁶, 4×10⁶, 5×10⁶, 6×10⁶, 7×10⁶, 8×10⁶, 9 ×10⁶, 10×10⁶, 11×10⁶, 12×10⁶, 13×10⁶, 14×10⁶, or 15×10⁶ cells.

Furthermore, the doses may be administered according to different regimens and timetables. For example, the disclosed methods can comprise an infusion, administration, or injection once a day, once every two days, once every three days, once every four days, once every five days, once every six days, a week, once every two weeks, once every three weeks, once every four weeks, once a month, once every other month, once every three months, or once every six months. In some embodiments, the disclosed methods can comprise continuous infusion, for instance, from a wearable pump. Similarly, the total time course of treatment may be about 5 weeks, about 10 weeks, about 15 weeks, about 20 weeks, about 25 weeks, about 30 weeks, about 35 weeks, about 40 weeks, about 45 weeks, about 50 weeks, about 55 weeks, about 60 weeks, about 65 weeks, about 70 weeks, about 75 weeks, or more. The patient may receive 1, 2, 3, 4, 5, 6, 7, 8, 9, 10, 11, 12, 13, 14, 15, 16, 17, 18, 19, or 20 or more infusions, administrations, or injections of monocytes over the course of treatment according to the disclosed methods.

As used herein, the term “intraventricular” refers to the space inside the ventricular system, specifically the cerebral ventricles. Accordingly, the term “intraventricular” and “intracerebroventricular” may be used interchangeably throughout this disclosure. Accordingly, “intraventricular administration” or “intraventricular injection” refers to delivery of a composition into the ventricals of the brain (i.e. the cerebral ventricles). The cerebral ventricles are a series of interconnected, fluid-filled spaces that lie in the core of the forebrain and brainstem. This system comprises four ventricles: the right and left lateral ventricles (one of which is found in each hemisphere of the brain), the third ventricle, and the fourth ventricle.

In accordance with certain aspects of the invention, an intrathecal delivery apparatus or an intracerebroventricular delivery apparatus may comprise a pump, fluid reservoir, monitoring system, a programmable control system, a catheter (such as an intrathecal or intracerebroventricular catheter), a battery and/or other elements known in the art.

Previous studies have indicated that repeated injection of primary monocytes via jugular vein is necessary to maintain the therapeutic effects in brain due to their short half-life (Lebson et al., 2010). Delivery route is a critical factor to consider when translating a therapeutic into the clinic. Venous access is the least invasive and most accepted by patients; however, it has a disadvantage that the transferred cells can be trapped in liver, spleen and lung after injection making the treatment inefficient (Lebson et al., 2010). Overcoming low therapeutic dose at target tissue by increasing the injected cell number raises concerns for thrombosis and cell lysis associated side effects. On the other hand, long-term intracerebroventricular (ICV) drug delivery systems, such as an Ommaya reservoir, have been regularly used in the clinic for decades in cancer patients requiring repeated dosing of chemotherapy in the CNS. Once the delivery system is implanted, the reservoir can be easily and safely accessed on the bedside (Cohen-Pfeffer et al., 2017). Based on the safety profile of these ICV delivery systems, coupled with the reduced concerns for thrombosis, cell lysis complications and off-target effects, delivery of the engineered CSR-monocytes via an ICV system for treatment is likely to achieve the most translational potential.

In some embodiments, a subject in need of treatment has an intraventricular catheter system having a reservoir and a catheter, such as Ommaya reservoir, implanted for ICV administration. In some embodiments, ICV administration is performed by injecting the aforementioned ICV formulations at a flow rate of 0.1-60 ml/minute, into the reservoir. In some embodiments, the cerebrospinal fluid (CSF) of a subject is drawn out at a flow rate of 0.1-60 ml/minute, from the reservoir before ICV administration of the formulations, so that there is no net increase in the CSF volume of the subject after ICV administration, to prevent pressure increase in the brain. In some embodiments, the formulation injected into the reservoir is allowed to travel through the catheter into the ventricle of a subject by gently pressing and releasing the reservoir.

### IV. Methods of Treatment

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies of the invention are used to delay development of a disease or to slow the progression of a disease.

In some embodiments, an engineered monocyte for use in treating a Tau protein associated disease or disorder is provided. In particular embodiments, an engineered monocyte for use in treating a tauopathy such as a neurodegenerative tauopathy is provided. Exemplary Tau protein associated diseases or disorders that can be treated that can be treated with engineered monocytes include, without limitation, Alzheimer's Disease, amyotrophic lateral sclerosis, Parkinson's disease, Creutzfeldt-Jacob disease, Dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, multiple system atrophy, Niemann-Pick disease, type C. Pallido-ponto-nigral degeneration, Pick's disease, progressive subcortical gliosis, progressive supranuclear palsy, Subacute sclerosing panencephalitis, Tangle only dementia, Postencephalitic Parkinsonism, and Myotonic dystrophy. In one embodiment, an engineered monocyte for use in treating Alzheimer's Disease (AD) is provided herein. Further, Tau protein associated diseases or disorders that can be treated with an engineered monocytes include diseases or disorders that are manifested in an impairment or loss of a cognitive function such as reasoning, situational judgement, memory capacity, learning, and/or special navigation. In certain embodiments, an engineered monocyte for use in a method of treatment is provided. In certain embodiments, the invention provides an engineered monocyte for use in a method of treating an individual, having any one of the Tau associated diseases or disorders described above, comprising administering to the individual an effective amount of the engineered monocytes. In one such embodiment, the method further comprises administering to the individual an effective amount of at least one additional therapeutic agent, e.g., as described below.

In certain embodiments, the invention provides an engineered monocyte for use in a method of reducing the levels of Tau protein (e.g., total Tau, total soluble Tau, soluble phosphorylated Tau, total insoluble Tau, insoluble phosphorylated Tau, hyperphosphorylated Tau, or paired helical filaments containing hyperphosphorylated Tau) in the brain (e.g., in the brain cortex and/or hippocampus) in an individual comprising administering to the individual an effective amount of the engineered monocytes to reduce the levels of Tau protein. An “individual” according to any of the above embodiments is a mammal, preferably a human

In some aspects, the invention provides a method for alleviating one or more symptoms of a Tau protein associated disease or disorder; or an engineered monocyte or a medicament comprising an engineered monocyte for alleviating one or more symptoms of a Tau protein associated disease or disorder (such as any of the diseases or disorders described herein, for example, AD). In some aspects, the invention provides a method for reducing the number of symptoms or the severity of one or more symptoms of a Tau protein associated disease or disorder; or an engineered monocyte or a medicament comprising an engineered monocyte for reducing the number of symptoms or the severity of one or more symptoms of a Tau protein associated disease or disorder (such as any of the diseases or disorders described herein, for example, AD). In a particular embodiment, the symptom of a Tau protein associated disease or disorder is an impairment in cognition. In a specific embodiment, the symptom of a Tau protein associated disease or disorder is an impairment in learning and/or memory. In a specific embodiment, the symptom of a Tau protein associated disease or disorder is a long-term memory loss. In a specific embodiment, the symptom of a Tau protein associated disease or disorder is dementia. In some embodiments, the symptom of a Tau protein associated disease or disorder is confusion, irritability, aggression, mood swings, or a language impairment. In some embodiments, the symptom of a Tau protein associated disease or disorder is an impairment or loss of one or more cognitive functions such as reasoning, situational judgment, memory capacity, and/or learning. The methods provided herein comprise administration of an amount (e.g., therapeutically effective amount) of engineered monocytes to an individual (e.g., who displays one or more symptoms of a Tau protein associated disease or disorder).

In specific aspects, the invention provides a method for retaining or increasing cognitive memory capacity, or for slowing down memory loss associated with a Tau protein associated disease or disorder; or an engineered monocyte or a medicament comprising an engineered monocyte for retaining or increasing cognitive memory capacity or for slowing down memory loss associated with a Tau protein associated disease or disorder (such as any of the diseases or disorders described herein, for example, AD). The methods provided herein comprise administration of an amount (e.g., therapeutically effective amount) of engineered monocytes to an individual (e.g., who displays one or more symptoms of memory loss or a decrease of memory capacity).

In some aspects, the invention provides a method for preventing the development of a Tau protein associated disease or disorder; or an engineered monocyte or a medicament comprising an engineered monocyte for preventing the development of a Tau protein associated disease or disorder (such as any of the diseases or disorders described herein, for example, AD). The methods provided herein comprise administration of an amount (e.g., therapeutically effective amount) of engineered monocytes to an individual (e.g., who is at risk of a Tau protein associated disease or disorder).

In some aspects, the invention provides a method for delaying the development of a Tau protein associated disease or disorder; or an engineered monocyte or a medicament comprising an engineered monocyte for delaying the development of a Tau protein associated disease or disorder (such as any of the diseases or disorders described herein, for example, AD). The methods provided herein comprise administration of an amount (e.g., therapeutically effective amount) of engineered monocytes to an individual (e.g., who displays one or more symptoms of a Tau protein associated disease or disorder).

In a further aspect, the invention provides pharmaceutical formulations comprising any of the engineered monocytes (e.g., monocytes expressing an anti-Tau CSR) provided herein, e.g., for use in any of the above therapeutic methods. In one embodiment, a pharmaceutical formulation comprises any of the engineered monocytes (e.g., monocytes expressing an anti-Tau CSR) provided herein and a pharmaceutically acceptable carrier. In another embodiment, a pharmaceutical formulation comprises any of the engineered monocytes (e.g., monocytes expressing an anti-Tau CSR) provided herein and at least one additional therapeutic agent, e.g., as described below.

Engineered monocytes of the invention can be used either alone or in combination with other agents in a therapy. For instance, an engineered monocyte of the invention may be co-administered with at least one additional therapeutic agent.

For example, the composition according to the invention may be administered in combination with other compositions comprising an biologically active substance or compound such as, for example, a known compound used in the medication of tauopathies and/or of amyloidoses, a group of diseases and disorders associated with amyloid or amyloid-like protein such as the amyloid protein involved in Alzheimer's Disease.

Generally, the other biologically active compound may include neutron-transmission enhancers, psychotherapeutic drugs, acetylcholine esterase inhibitors, calcium-channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-D-aspartate glutamate receptor antagonists, non-steroidal anti-inflammatory drugs, antioxidants, and serotonergic receptor antagonists. In particular, the biologically active agent or compound may comprise at least one compound selected from the group consisting of compounds against oxidative stress, anti-apoptotic compounds, metal chelators, inhibitors of DNA repair such as pirenzepin and metabolites, 3-amino-1-propanesulfonic acid (SAPS), 1,3-propanedisulfonate (1,3PDS), secretase activators, [bet]- and 7-secretase inhibitors, tau proteins, neurotransmitter, /3-sheet breakers, antiinflammatory molecules, “atypical antipsychotics” such as, for example clozapine, ziprasidone, risperidone, aripiprazole or olanzapine or cholinesterase inhibitors (ChEIs) such as tacrine, rivastigmine, donepezil, and/or galantamine and other drugs and nutritive supplements such as, for example, vitamin B 12, cysteine, a precursor of acetylcholine, lecithin, choline, Ginkgo biloba, acyetyl-L-carnitine, idebenone, propentofylline, or a xanthine derivative, together with an engineered monocyte the invention including and, optionally, a pharmaceutically acceptable carrier and/or a diluent and/or an excipient and instructions for the treatment of diseases.

In a further embodiment, the composition according to the invention may comprise niacin or memantine together with an engineered monocyte according to the invention and, optionally, a pharmaceutically acceptable carrier and/or a diluent and/or an excipient.

In still another embodiment of the invention compositions are provided that comprise “atypical antipsychotics” such as, for example clozapine, ziprasidone, risperidone, aripiprazole or olanzapine for the treatment of positive and negative psychotic symptoms including hallucinations, delusions, thought disorders (manifested by marked incoherence, derailment, tangentiality), and bizarre or disorganized behavior, as well as anhedonia, flattened affect, apathy, and social withdrawal, together with the engineered monocytes of the invention and, optionally, a pharmaceutically acceptable carrier and/or a diluent and/or an excipient.

Other compounds that can be suitably used in compositions in addition to engineered monocytes according to the invention, are those disclosed, for example, in WO 2004/058258 (see especially pages 16 and 17) including therapeutic drug targets (page 36-39), alkanesulfonic acids and alkanolsulfuric acid (pages 39-51), cholinesterase inhibitors (pages 51-56), NMDA receptor antagonists (pages 56-58), estrogens (pages 58-59), non-steroidal anti-inflammatory drugs (pages 60-61), antioxidants (pages 61-62), peroxisome proliferators-activated receptors (PPAR) agonists (pages 63-67), cholesterol-lowering agents (pages 68-75); amyloid inhibitors (pages 75-77), amyloid formation inhibitors (pages 77-78), metal chelators (pages 78-79), anti-psychotics and anti-depressants (pages 80-82), nutritional supplements (pages 83-89) and compounds increasing the availability of biologically active substances in the brain (see pages 89-93) and prodrugs (pages 93 and 94), which document is incorporated herein by reference, but especially the compounds mentioned on the pages indicated above.

Such combination therapies noted above encompass combined administration (where two or more therapeutic agents are included in the same or separate formulations), and separate administration, in which case, administration of the engineered monocytes of the invention can occur prior to, simultaneously, and/or following, administration of the additional therapeutic agent or agents. In one embodiment, administration of the engineered monocytes and administration of an additional therapeutic agent occur within about one month, or within about one, two or three weeks, or within about one, two, three, four, five, or six days, of each other.

An engineered monocyte of the invention (and any additional therapeutic agent) can be administered by any suitable means, including intrathecal and intraventricular injection. Various dosing schedules including but not limited to single or multiple administrations over various time-points, bolus administration, and pulse infusion are contemplated herein.

Engineered monocytes of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The engineered monocytes need not be, but are optionally formulated with one or more agents currently used to prevent or treat the disorder in question. The effective amount of such other agents depends on the amount of engineered moonocytes present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

### V. Kits

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be a vial having a stopper pierceable by a hypodermic injection needle). At least one active agent in the composition is an engineered monocyte of the invention. The label or package insert indicates that the composition is used for treating the condition of choice. Moreover, the article of manufacture may comprise (a) a first container with a composition contained therein, wherein the composition comprises an engineered monocyte of the invention; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. In addition, or alternatively, the article of manufacture may comprise a nucleic acid encoding a chimeric scavenger receptor of the invention. Such nucleic acid may be a viral vector. It may further include other materials desirable from a commercial and user standpoint, including other buffers, diluents, filters, needles, and syringes.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Antibody-Redirected Chimeric Scavenger Receptors (CSR)

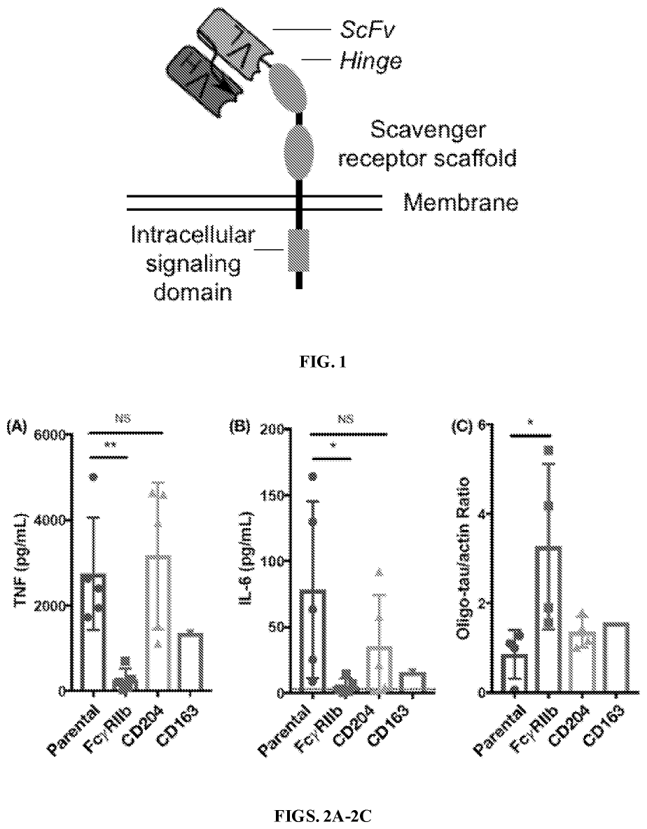

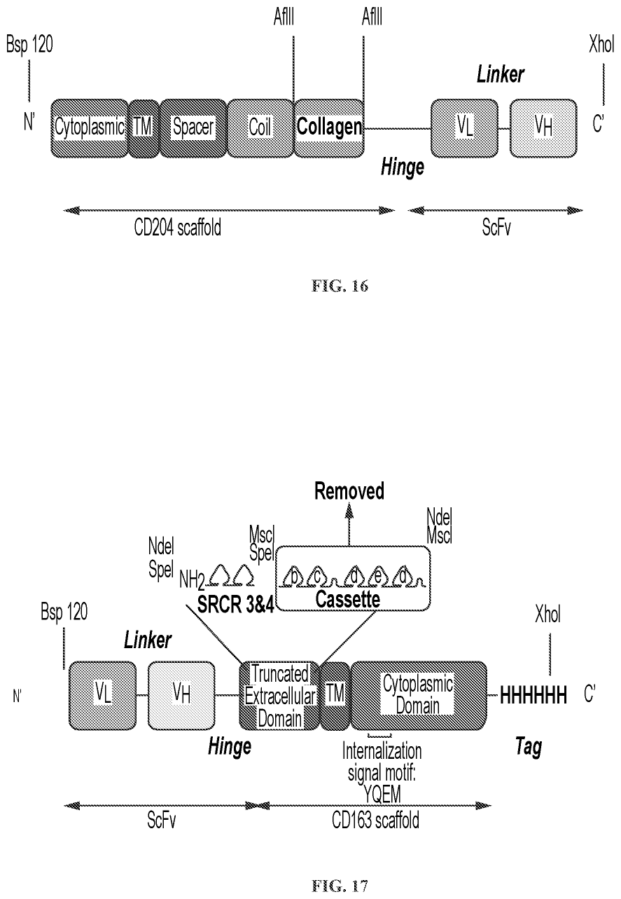

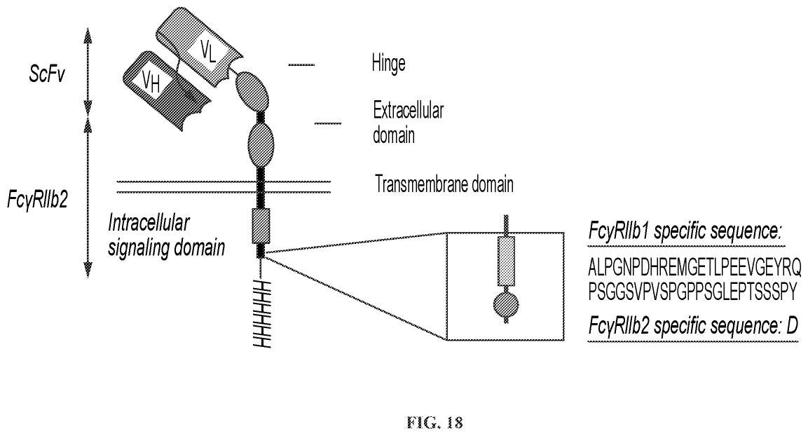

Scavenger receptors can attenuate inflammatory responses of myeloid cells in the cancer microenvironment. FcγRIIb, CD163, and CD204 on myeloid cells have been shown to dampen inflammatory cytotoxicity against various types of tumors (Clynes et al., 2000; Yu et al., 2015). These anti-inflammatory properties were harnessed to reprogram the inherent inflammatory myeloid response to o-pTau in AD. Monocytes engineered to bind and internalize o-pTau via antibody-redirected chimeric scavenger receptors (CSR) will halt the progression of AD by protecting neurons from o-pTau-mediated neurotoxicity while dampening proinflammatory cytokine release. Three CSRs were designed by replacing the natural ligand-binding domain of the scavenger receptors with an anti-o-pTau single-chain variable fragment (ScFv) based on an in vivo validated anti-o-pTau antibody (Lee et al., 2016; U.S. Pat. No. 9,598,485). These three CSR constructs encoding an anti-o-pTau ScFv supported by an FcγRIIb (FIG. 14), CD163 (FIG. 15), or CD204 (FIG. 16) scaffold (FIG. 1) were cloned into antibiotic selectable retroviral vectors. For the CD163-based construct, a truncated extracellular domain was used that consists of SRCR3-9 (FIG. 15). In addition, a modified CD163-based construct was generated by removing the long-range cassette (SCRCS-9) from the extracellular domain (FIG. 17). For both the CD163- and CD204-based construct, the natural ligand binding domain was replaced by the ScFv sequence. For the FcγRIIb-based construct, the ScFc was added to the entirety of FcγRIIb (FIG. 14). In addition, a modified FcγRIIb-based construct was generated by replacing a portion of the FcγRIIb1 intracellular signaling domain sequence was the FcγRIIb2 intracellular signaling domain sequence (FIG. 18).

### Example 2—CSR-Engineered Monocytes

The murine monocyte/macrophage cell line PMJ2-PC was retrovirally transduced with each of the vectors described in Example 1, and stable CSR-expressing cell lines were obtained via drug selection.

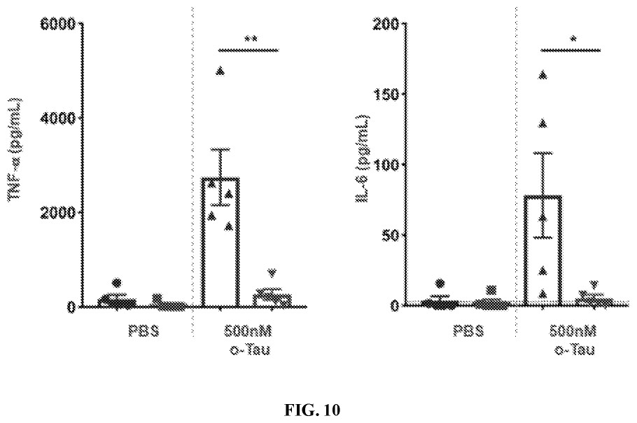

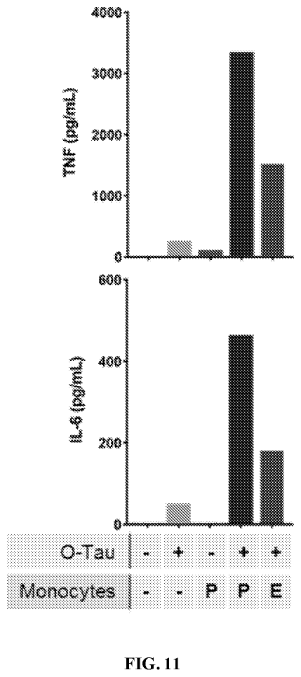

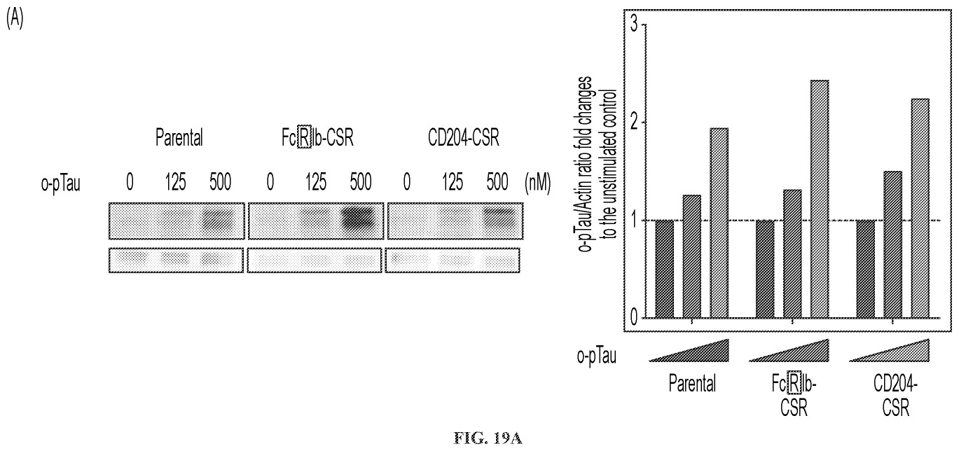

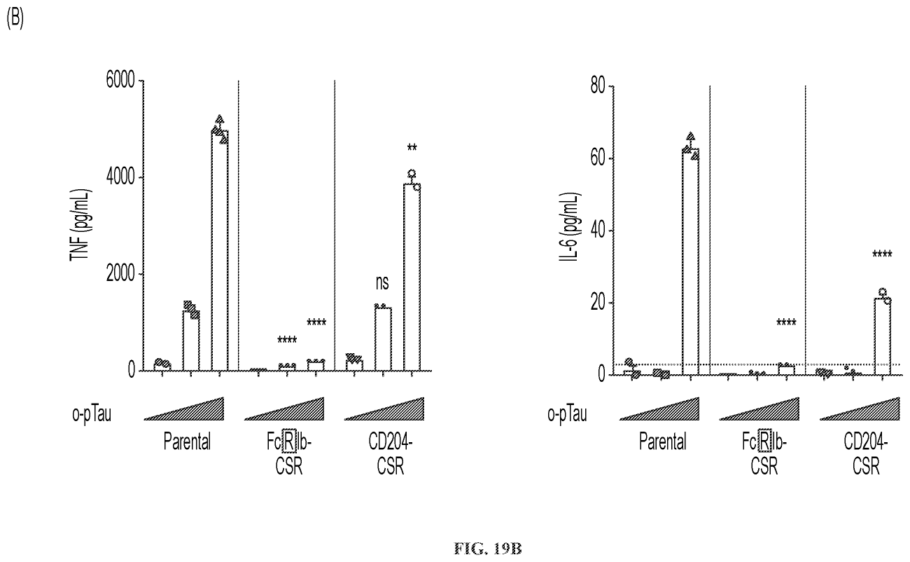

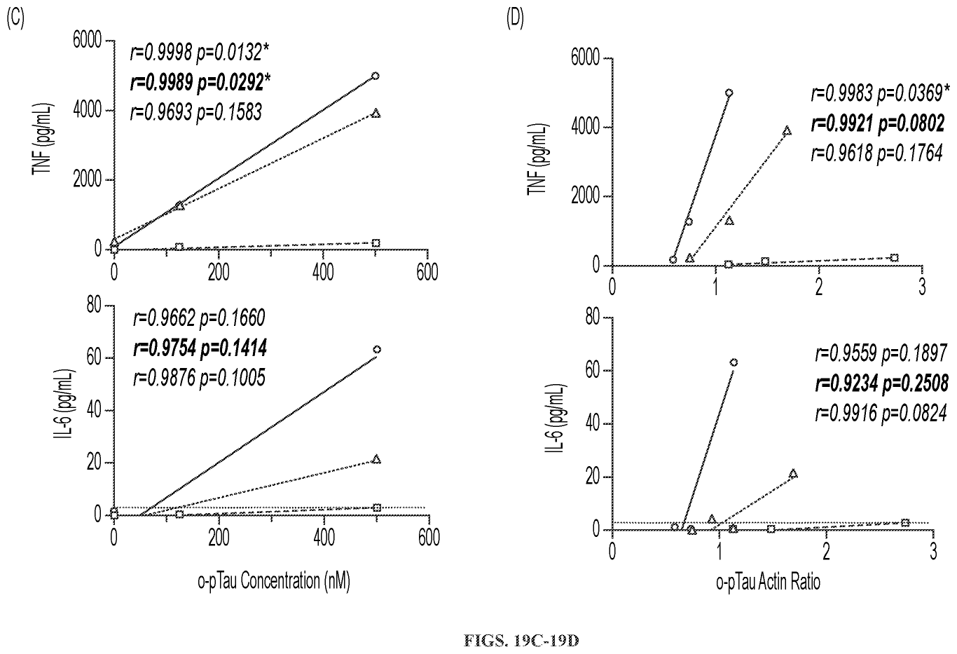

Each of the engineered CSR-monocyte lines trended toward enhanced in vitro uptake of o-pTau and reduced proinflammatory cytokine production upon o-pTau stimulation (FIGS. 2A-2C and 19C). Among these constructs, the FcγRIIb CSR monocytes demonstrate the most limited release of inflammatory cytokines (FIGS. 2A, 2B, 10, 11, 19B) combined with the most significant increase in cell-associated o-pTau (FIG. 2C and 19A).

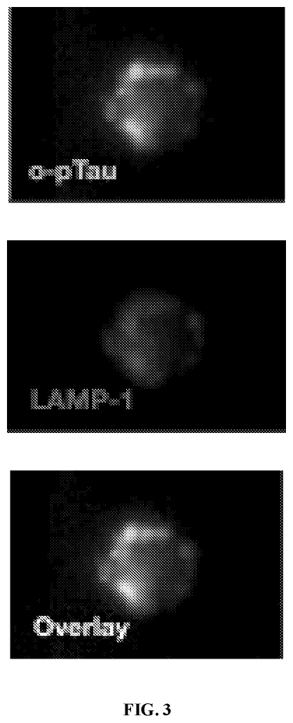

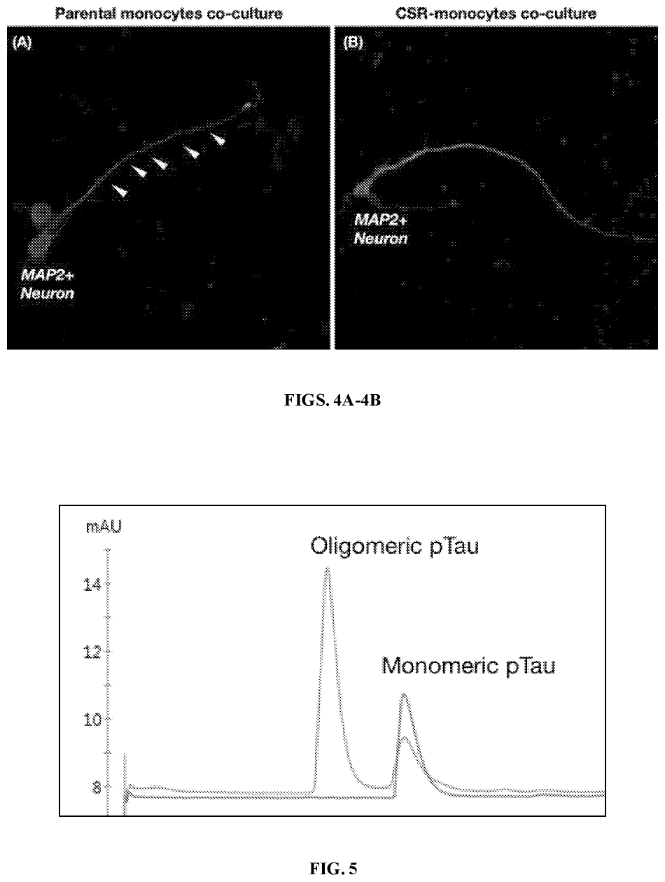

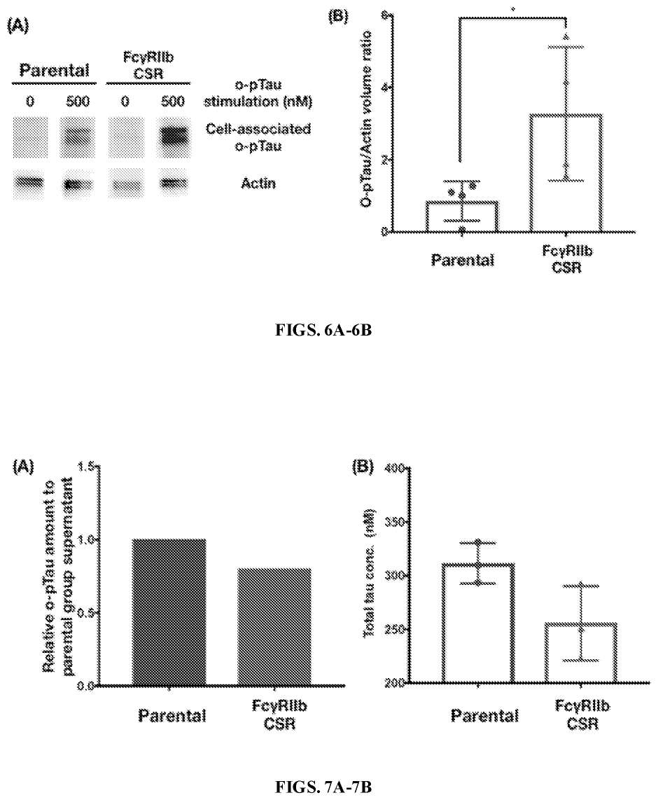

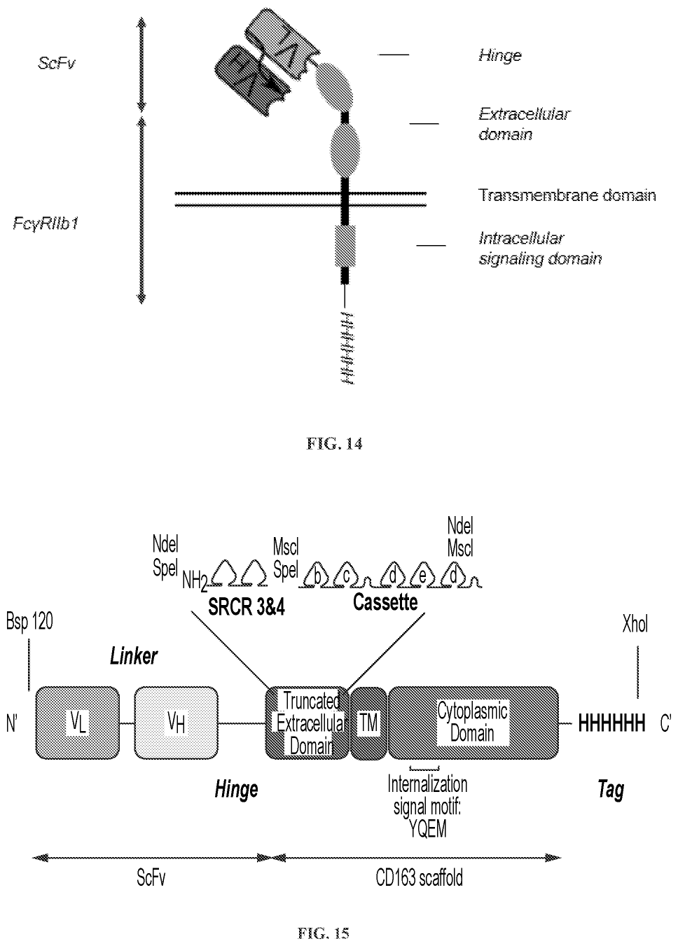

FcγRIIb-CSR monocytes can internalize o-pTau, and most of the cell-associated o-pTau on FcγIIb-CSR-monocytes is intracellular (FIG. 13). In addition, the internalized o-pTau co-localizes with lysosomes (FIG. 3). Furthermore, FcγRIIb-CSR monocytes have higher o-pTau binding (FIGS. 6A, 6B, 19A, and 19D) and are more capable of clearing o-pTau in vitro compared to parental monocytes (FIG. 7). FcγRIIb-CSR monocytes can also protect neurons from degradation in the presence of o-pTau in vitro (FIG. 4).

### Example 3—Internalization of o-pTau by CSR-Engineered Monocytes

CSR-monocytes have higher o-pTau uptake, which suggests that CSR-monocytes may utilize an internalization mechanism distinct from macropinocytosis used by parental monocytes. Previous literature has indicated that the internalization pathway of unmodified FcγRIIb is dependent on the size of the ligand bound to the receptor. FcγRIIb is internalized via clathrin-mediated endocytosis if its ligand is<1 μm in diameter or via phagocytosis if the ligand is>3 μm in diameter, where phagocytosis in an actin dependent process and clathrin-mediated endocytosis is mediated by clathrin and dynamin but generally not by actin (Miettinen et al., 1989; Tse et al., 2003). Despite the fact that o-pTau is estimated at˜20 nm in diameter (Xu et al., 2010), the modified structure of FcγRIIb-CSR may result in a differential internalization pathway in monocytes than its unmodified counterpart. The CSR-transduced monocytes may use clathrin-mediated endocytosis to facilitate internalization of o-pTau.

To examine this, recombinant o-pTau is generated from pTau monomers, isolated utilizing size-exclusion chromatography (FIG. 5), and passed through a 0.2 μm filter to remove potential microbial contamination. For imaging studies, o-pTau is further conjugated with fluorophore (either Alexa or Qdot). The recombinant o-pTau tagged with a fluorophore will be added to CSR-monocytes pre-incubated with a clathrin inhibitor (e.g. Pitstop2 or monodansylcadaverine (von Kleist et al., 2011; Schlegel et al., 1982)) or an Actin inhibitor with blocks phagocytosis but not always clathrin-mediated endocytosis (Latrunculin A) before stimulation with fluorophore-labeled o-pTau. If internalization is clathrin-driven, the first two inhibitors and possibly the third will block it; however, if it is phagocytosis driven, only the last will. The control group will be pre-incubated with phosphate buffer saline (PBS). A pulse-chase experiment will be performed to determine the kinetics of the internalization process, for which the CSR-monocytes or parental monocytes will be stimulated with fluorescently labeled o-pTau on ice and wash away the unbound o-pTau. Next, the cellular internalization activity will be tracked by moving the cells from ice to 37° C. incubation for 15, 30, 60 and 120 minutes (Miettinen et al., 1989). The cells will then be fixed and their plasma membranes stained for evaluation of their o-pTau internalization by Amnis® imaging flow cytometry and confocal microscopy. The percentage of the internalized fluorescence signal over the total cellular associated fluorescence signal within the cytoplasm over the total cellular associated fluorescence signal is defined as the o-pTau internalization activity. Parental PMJ2-PC will act as a negative control for examining the basal internalization efficiency of FcγRIIb.

In the PBS treated CSR-monocyte group, o-pTau internalization activity is expected to begin to rise as early as 15 minutes, and reach a plateau near 120 minutes (Miettinen et al., 1989). Because the CSR-monocytes have enhanced o-pTau binding compared to parental monocytes, the o-pTau internalization activity at any given time point is expected to be higher in the CSR-monocytes than in the parental monocytes. When the CSR-monocytes are pre-incubated with clathrin inhibitors, the fluorescence signal is expected to mostly localize to the plasma membrane but not inside the cells indicating that the o-pTau internalization activity is hindered, and suggesting that clathrin-mediated endocytosis is necessary for CSR mediated o-pTau internalization. Notably, clathrin inhibitors may not hamper the internalization activity in the parental monocytes because the basal uptake of the pTau assemblies reportedly occurs via micropinocytosis (Holmes et al., 2013; Mirbaha et al., 2015).

Although clathrin-dependent endocytosis is the major pathway for receptor-mediated endocytosis, it is also possible that CSR internalize through clathrin-independent pathways. An alternative mechanism could be receptor-mediated phagocytosis; however, this would be detected using Latrunculin (Fujimoto et al., 2000). The caveolar pathway is a clathrin-independent pathway for receptor-mediated endocytosis which might also be blocked by Latrunculin (Mayor et al., 2014). Discrimination of the caveolar pathway from phagocytosis in CSR internalization can be examined by pre-treatment of the CSR-monocytes with the siRNA of Caveolin, which is the critical component of the pathway (Ge et al., 2004). Another alternative mechanism could be receptor-mediated phagocytosis which is a pathway utilized by monocytes/macrophages and can be determined by pre-incubation of CSR-monocytes with a phagocytosis inhibitor like Cytochalasin D or latrunculin (Fujimoto et al., 2000).

FcγRIIb1-CSR and FcγRIIb2-CSR constructs cloned into the pMG-eYFP retroviral vector were overexpressed in a murine monocyte/macrophage cell line, PMJ2PC. The PMJ2PC transduced with the pMG-eYFP vector without inserts served as the control monocytes. Successfully transduced monocytes were enriched using flow cytometry. The monocytes were incubated with 500 nM of o-Tau at 37° C. for 24 hours before we evaluated their TNF secretion and o-Tau uptake level by quantifying the TNF concentration of the supernatant using ELISA and the cell-associated o-Tau amount in the cell lysates using Western Blot.

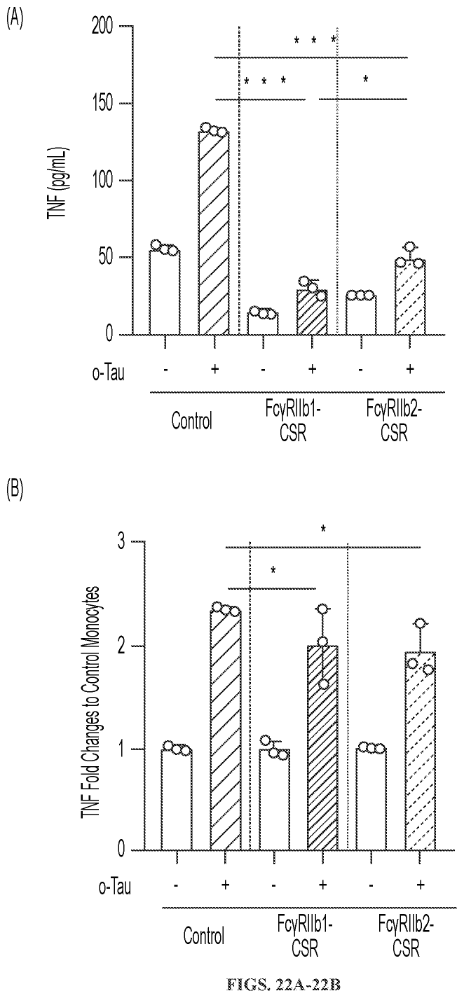

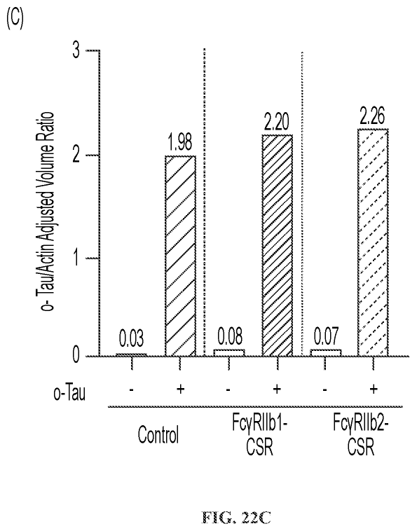

Compared to the Control monocytes, both the FcγRIIb1-CSR and FcγRIIb2-CSR monocytes have a lower o-Tau induced TNF expression (FIGS. 22A and 22B), which indicated these two CSRs both capable of dampening proinflammatory responses in monocytes to o-Tau. Although the TNF level was below the suggested detection limit, 80 pg/mL, the FcγRIIb1-CSR monocyte might have a lower o-Tau induced TNF secretion than the FcγRIIb2-CSR monocyte. This finding indicated that FcγRIIb1-CSR has a greater potential than FcγRIIb2-CSR in limiting the inflammatory responses to o-Tau stimulation. Furthermore, cell-associated o-Tau of both FcγRIIb1-CSR and FcγRIIb2-CSR monocytes are higher than the control monocytes (FIG. 22C), which indicated an enhanced capability of both CSRs in removing o-Tau from the surroundings. In conclusion, both the FcγRIIb1-CSR and FcγRIIb2-CSR demonstrated a reduced proinflammatory response and an enhanced o-Tau extraction from the environment.

### Example 4—Degradation of o-pTau in CSR-Engineered Monocytes

Ligand-bound FcγRIIb is transported to the lysosomal compartment for degradation (Miettinen et al., 1989; Tse et al., 2003). As shown here, o-pTau co-localizes with lysosomes (FIG. 3), and lysosomal activity is enhanced as measured by LAMP-1 staining (Yu et al., 2010) in CSR-monocytes upon stimulation with o-pTau (FIG. 8). Based on this evidence, o-pTau internalized via FcγRIIb-CSR may be degraded via lysosomes in the engineered monocytes.

To further verify the co-localization of internalized o-pTau-bound FcγRIIb-CSR complex and lysosomes (FIG. 3), CSR-monocytes will be pulse stimulated with fluorophore-labeled o-pTau and the excess washed away. The cells will be incubated at 37° C. for three hours, which is the estimated half-life for internalized substances, before collection for fixation and staining (Mellman et al., 1983). The lysosomes will be stained using anti-LAMP-1 antibody, and endosomes stained using anti-Rab7 antibody. The co-localization of these compartments will be determined by Amnis® imaging flow cytometry and by confocal microscopy.

To evaluate the necessity of lysosomes in degradation of the endocytosed o-pTau, CSR-monocytes will be pre-treated with a lysosome inhibitor (e.g. choloroquine or bafilomycin A (Wang et al., 1993; Yamamoto et al., 1998)), prior to pulse stimulation with fluorophore-labeled o-pTau. The control will be CSR-monocytes pre-treated with PBS. The CSR-monocytes will be either fixed after three hours at 37° C. incubation or without incubation (Mellman et al., 1983). The intensity of the intracellular fluorescence from o-pTau will be assessed using Amnis® imaging flow cytometry and by confocal microscopy. The degradation efficiency will be evaluated by the ratio of the intensity of the incubated group (T1) over the group without incubation (T0). Lower T1/T0 ratio indicates a more active o-pTau degradation.

The co-localization of o-pTau, LAMP-1, and Rab7, which would suggest that the o-pTau is being processed by the endolysosomal system, is expected. The administration of lysosome inhibitors is expected to result in a higher T1/T0 ratio, suggesting an impaired degradation of o-pTau. Together, these data will establish whether the lysosomal pathway is necessary for degradation of internalized o-pTau.

However, if the signal of o-pTau and LAMP-1 do not co-localize or the degradation efficiency, T1/T0 ratio, is not changed in the lysosome inhibitor treatment group, it would indicate that lysosomes are not necessary for the degradation of the internalized o-pTau and that o-pTau is degraded by an alternative pathway. Previous literature shows that the proteasomal pathway is also involved in the degradation of ligand-bound FcγR (Marois et al., 2009). To determine whether the proteasomal system is necessary for the internalized o-pTau degradation, the proteasomal activities will be inhibited in the CSR-monocytes with a proteasome inhibitor like lactacystin or MG132 (Fenteany et al., 1995; Lee & Goldberg, 1996) and o-pTau degradation efficiency examined If the proteasomal system is required, then a lower T1/T0 ratio will be observed. If no loss of internalized opTau fluorescence is observed over time in the absence of any inhibitors, then opTau is not being degraded but rather accumulated in an intracellular organelle.

### Example 5—CSR-Engineered Monocytes Halt the Progression of AD

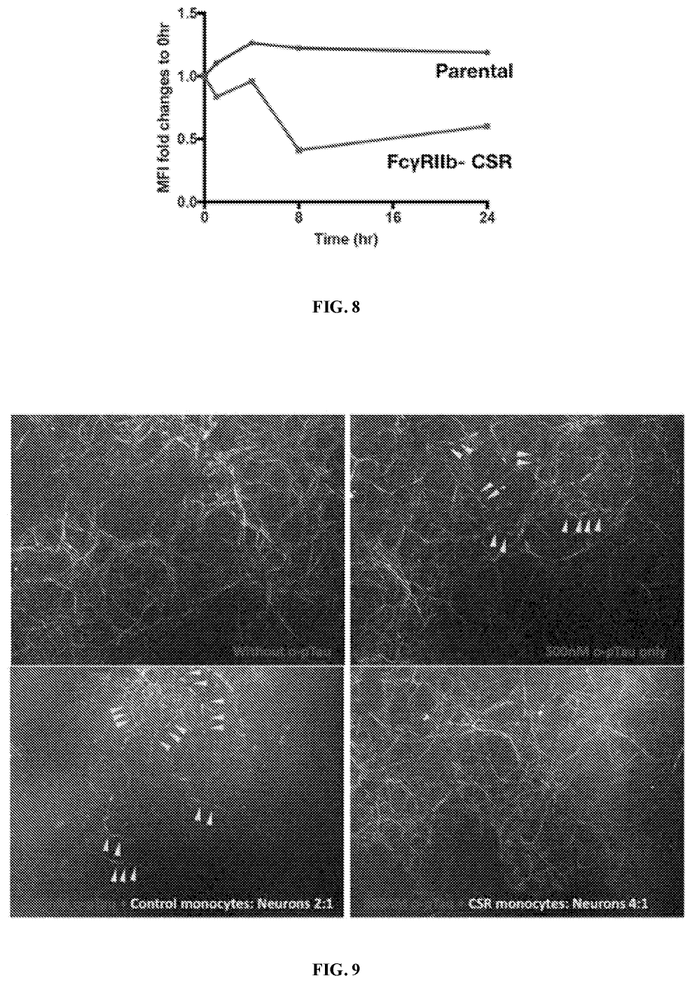

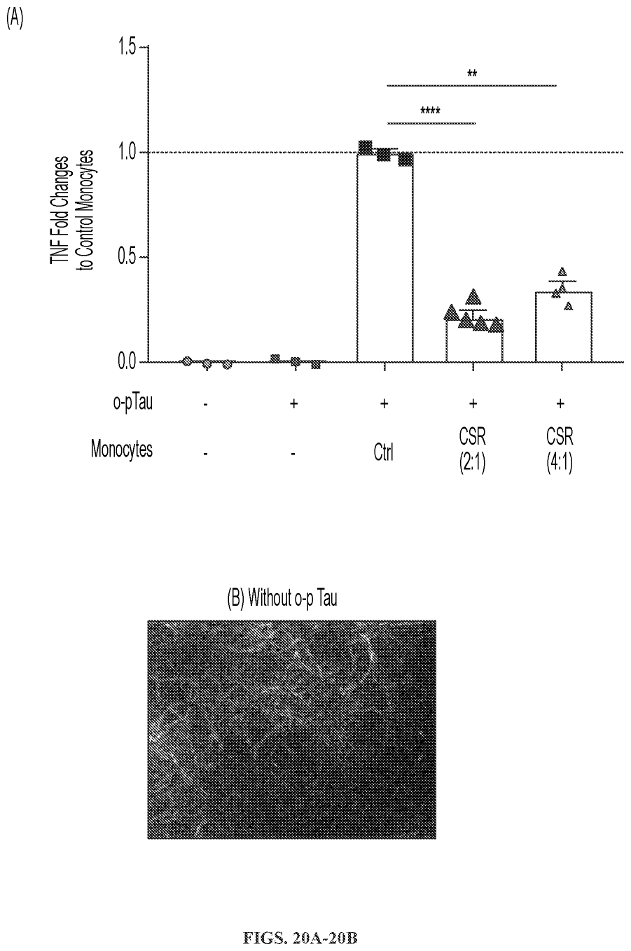

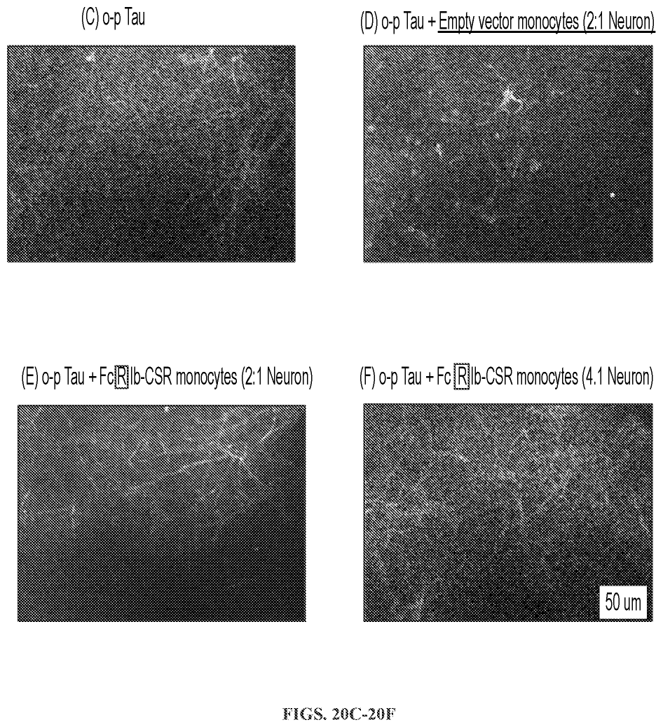

o-pTau assemblies as well as proinflammatory cytokines in the CNS were proven to be detrimental to neurons and lead to cognitive decline (Karran et al., 2011; Nelson et al., 2009; Braak & Braak, 1991; Maphis et al., 2015; Ghosh, 2013; Kitazawa et al., 2011; Li et al., 1997; Bellinger et al., 1995). The CSR-monocytes have shown a favorable in vitro profile including enhanced o-pTau clearance and reduced proinflammatory cytokine production (FIGS. 2, 6, and 7). To further evaluate the protective capability of the CSR-monocytes, a co-culture system has been developed in which primary hippocampal neurons are utilized to assess neuronal damage mediated by o-pTau and proinflammatory cytokines. Primary hippocampal neurons were isolated from E18 C57BL6 pups and cultured in a specially coated cover-slide or plate (Seibenhener & Wooten, 2012). After the primary neurons are cultured in vitro for 14 days, they are stimulated with o-pTau with either CSR-monocytes or control monocytes for three days. The co-culture system is then fixed and stained for microtubule associated protein 2 (MAP2), a marker for neuronal dendrites. Neuronal dendrites sustain apparent damage in the course of AD pathology (Knafo et al., 2009) and fragmentation of MAP2 staining has been utilized to visualize this neuronal damage (Lee et al., 2016). The slides are next imaged using confocal microscopy (FIG. 3). The plates are imaged using Operetta, a high-content imaging system, for quantification and statistical analysis. Primary neurons had significantly better MAP2 integrity when they were co-cultured with CSR-monocytes, compared to co-culture with control monocytes (FIGS. 9 and 20B-F). Furthermore, the CSR-monocytes secreted significantly lower levels of TNF than control monocytes (FIG. 20A).

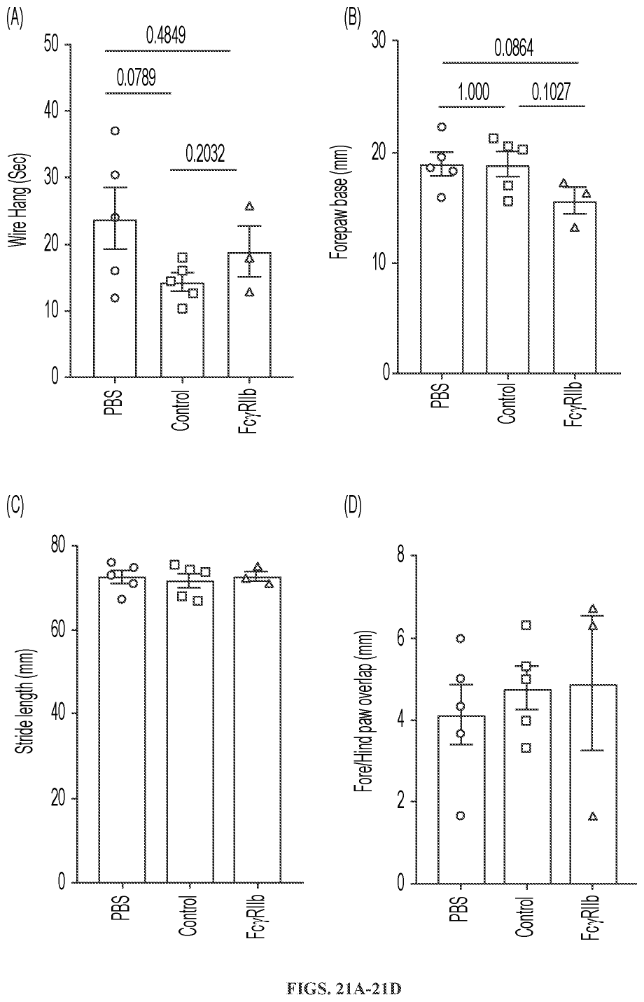

In addition, inhibition of inflammation and reduction of pTau burden in the brain have been shown to result in cognitive improvement in mouse models. Treatment with CSR-monocytes in vivo is expected to reduce pTau burden and limit proinflammatory cytokine production in the brain, improving cognitive function in an AD patients. To this end, in vivo experiments were performed to evaluate therapeutic efficacy. For the in vivo experiments, ICV access was established to the lateral ventricle by implanting an infusion catheter and port (DeVos & Miller, 2013) in P301S Tau overexpressing transgenic mice, a commonly used AD mouse model (Yoshiyama et al., 2017; Takeuchi et al., 2011). Next, primary monocytes overexpressing FcγRIIb-CSR were infused via the port for the treatment group. The control groups were mice receiving PBS injection and mice receiving injection with control monocytes. The mice underwent weekly treatment for two months starting when they were 4-months old. The capability of the CSR-monocytes to affect wire hang (FIG. 21A), forepaw base (FIG. 21B), stride length (FIG. 21C), and fore/hind paw overlap (FIG. 21D) was analyzed.

Additional in vivo experiments will be performed to evaluate therapeutic efficacy. For the in vivo experiments, ICV access will be established to the lateral ventricle by implanting an infusion catheter and port (DeVos & Miller, 2013) in P301S Tau overexpressing transgenic mice, a commonly used AD mouse model (Yoshiyama et al., 2017; Takeuchi et al., 2011). Next, primary monocytes overexpressing FcγRIIb-CSR will be infused via the port for the treatment group. The control groups will be mice receiving no injection and mice receiving injection with parental primary monocytes or those transduced with empty vectors. The mice will start weekly treatment for two months when they are 6-months old, when Tau pathology but not neuronal loss becomes evident (Lee et al., 2016; Toshiyama et al., 2007). The capability of the CSR-monocytes to halt progressions of 1) Tau pathology and neuronal loss, 2) inflammation in the CNS and 3) cognitive decline will be assessed.

The object recognition task will be used to assess cognitive functions at one month and two months post treatment (Lasagna-Reeves et al., 2011). This two-session test allows the mice to be exposed to two objects in a defined space in the first session and their time spent exploring the objects will be recorded. In the second session, a novel object will replace one of the objects. The mice will be placed into the defined space and the time exploring on the novel object will be the indicator for their recognition-associated memory

(Leger et al., 2013). Their brain tissues will also be collected after two months of treatment for immunofluorescence confocal microscopic examination and the brain homogenate will be analyzed for an inflammatory transcription profile by RNA sequencing.

The mice in the treatment group are expected to outperform the control groups in object recognition tasks by showing more time spent on the novel object. Since the neuronal loss is evident at the age of 8 months in this P301S mouse model (Yoshiyama et al., 2007), reduced neuronal loss and Tauopathy in the treatment group compared to the control groups is expected by the time brain pathology is examined Based on the reduced proinflammatory profile of CSR-monocytes in vitro, the inflammatory transcription profile in the brain homogenate is expected to also demonstrate a lower inflammatory signature in the treatment group.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Bellinger F P, Madamba S G, Campbell I L, Siggins G R. Reduced long-term potentiation in the dentate gyms of transgenic mice with cerebral overexpression of interleukin-6. Neurosci Lett. 1995; 198(2):95-98.

Bolos M, Llorens-Martin M, Perea J R, et al. Absence of CX3CR1 impairs the internalization of Tau by microglia. Mol Neurodegener. 2017; 12(1):59.

Bolos M, Llorens-Martin M, Jurado-Arjona J, Hernandez F, Rabano A, Avila J. Direct Evidence of Internalization of Tau by Microglia In Vitro and In Vivo. J Alzheimers Dis. 2016; 50(1):77-87.

Braak H, Braak E. Neuropathological stageing of Alzheimer-related changes. Acta Neuropathol. 1991; 82(4):239-259.

Clynes R A, Towers T L, Presta L G, Ravetch J V. Inhibitory Fc receptors modulate in vivo cytotoxicity against tumor targets. Nat Med. 2000; 6(4):443-446.

Cohen-Pfeffer J L, Gururangan S, Lester T, et al. Intracerebroventricular Delivery as a Safe, Long-Term Route of Drug Administration. Pediatr Neurol. 2017; 67:23-35.

DeVos S L, Miller T M. Direct intraventricular delivery of drugs to the rodent central nervous system. J Vis Exp. 2013(75):e50326.

Fenteany G, Standaert R F, Lane W S, Choi S, Corey E J, Schreiber S L. Inhibition of proteasome activities and subunit-specific amino-terminal threonine modification by lactacystin. Science. 1995; 268(5211):726-731.

Fiala M, Zhang L, Gan X, et al. Amyloid-beta induces chemokine secretion and monocyte migration across a human blood—brain barrier model. Mol Med. 1998; 4(7):480-489.

Fujimoto L M, Roth R, Heuser J E, Schmid S L. Actin assembly plays a variable, but not obligatory role in receptor-mediated endocytosis in mammalian cells. Traffic. 2000; 1(2):161-171.

Ge S, Pachter J S. Caveolin-1 knockdown by small interfering RNA suppresses responses to the chemokine monocyte chemoattractant protein-1 by human astrocytes. J Biol Chem. 2004; 279(8):6688-6695.

Ghosh S, Wu M D, Shaftel S S, et al. Sustained interleukin-1beta overexpression exacerbates tau pathology despite reduced amyloid burden in an Alzheimer's mouse model. J Neurosci. 2013; 33(11):5053-5064.

Group A-F R. Follow-up evaluation of cognitive function in the randomized Alzheimer's Disease Anti-inflammatory Prevention Trial and its Follow-up Study Alzheimers Dement. 2015; 11(2):216-225 e211.

Hebert L E, Weuve J, Scherr P A, Evans D A. Alzheimer disease in the U.S. (2010-2050) estimated using the 2010 census. Neurology. 2013; 80(19):1778-1783.

Heppner F L, Ransohoff R M, Becher B. Immune attack: the role of inflammation in Alzheimer disease. Nat Rev Neurosci. 2015; 16(6):358-372.

Holmes B B, DeVos S L, Kfoury N, et al. Heparan sulfate proteoglycans mediate internalization and propagation of specific proteopathic seeds. Proc Natl Acad Sci USA. 2013; 110(33):E3138-3147.

Karran E, Mercken M, De Strooper B. The amyloid cascade hypothesis for Alzheimer's disease: an appraisal for the development of therapeutics. Nat Rev Drug Discov. 2011; 10(9):698-712.

Kitazawa M, Cheng D, Tsukamoto M R, et al. Blocking IL-1 signaling rescues cognition, attenuates tau pathology, and restores neuronal beta-catenin pathway function in an Alzheimer's disease model. J Immunol. 2011; 187(12):6539-6549.

Knafo S, Alonso-Nanclares L, Gonzalez-Soriano J, et al. Widespread changes in dendritic spines in a model of Alzheimer's disease. Cereb Cortex. 2009; 19(3):586-592.

Lal S, Lacroix M, Tofilon P, Fuller G N, Sawaya R, Lang F F. An implantable guide-screw system for brain tumor studies in small animals. J Neurosurg. 2000; 92(2):326-333.

Lasagna-Reeves C A, Castillo-Carranza D L, Sengupta U, Clos A L, Jackson G R, Kayed R. Tau oligomers impair memory and induce synaptic and mitochondrial dysfunction in wild-type mice. Mol Neurodegener. 2011; 6:39.

Lebson L, Nash K, Kamath S, et al. Trafficking CD11b-positive blood cells deliver therapeutic genes to the brain of amyloid-depositing transgenic mice. J Neurosci. 2010; 30(29):9651-9658.

Lee S H, Le Pichon C E, Adolfsson O, et al. Antibody-Mediated Targeting of Tau In Vivo Does Not Require Effector Function and Microglial Engagement. Cell Rep. 2016; 16(6):1690-1700.

Lee D H, Goldberg A L. Selective inhibitors of the proteasome-dependent and vacuolar pathways of protein degradation in Saccharomyces cerevisiae. J Biol Chem. 1996; 271(44):27280-27284.

Leger M, Quiedeville A, Bouet V, et al. Object recognition test in mice. Nat Protoc. 2013; 8(12):2531-2537.

Li A J, Katafuchi T, Oda S, Hori T, Oomura Y. Interleukin-6 inhibits long-term potentiation in rat hippocampal slices. Brain Res. 1997; 748(1-2):30-38.

Majerova P, Michalicova A, Cente M, et al. Trafficking of immune cells across the blood-brain barrier is modulated by neurofibrillary pathology in tauopathies. PLoS One. 2019; 14(5):e0217216.

Majerova P, Zilkova M, Kazmerova Z, et al. Microglia display modest phagocytic capacity for extracellular tau oligomers. J Neuroinflammation. 2014; 11:161.

Maphis N, Xu G, Kokiko-Cochran O N, et al. Reactive microglia drive tau pathology and contribute to the spreading of pathological tau in the brain. Brain. 2015; 138(Pt 6):1738-1755.

Marois L, Vaillancourt M, Marois S, et al. The ubiquitin ligase c-Cbl down-regulates FcgammaRIIa activation in human neutrophils. J Immunol. 2009; 182(4):2374-2384.

Mayor S, Parton R G, Donaldson J G. Clathrin-independent pathways of endocytosis. Cold Spring Harb Perspect Biol. 2014; 6(6).

Medina M. An Overview on the Clinical Development of Tau-Based Therapeutics. Int J Mol Sci. 2018; 19(4).

Mellman I S, Plutner H, Steinman R M, Unkeless J C, Cohn Z A. Internalization and degradation of macrophage Fc receptors during receptor-mediated phagocytosis. J Cell Biol. 1983; 96(3):887-895.

Meyer PF, Tremblay-Mercier J, Leoutsakos J, et al. INTREPAD: A randomized trial of naproxen to slow progress of presymptomatic Alzheimer disease. Neurology. 2019; 92(18):e2070-e2080.

Miettinen H M, Rose J K, Mellman I. Fc receptor isoforms exhibit distinct abilities for coated pit localization as a result of cytoplasmic domain heterogeneity. Cell. 1989; 58(2):317-327.

Mirbaha H, Holmes B B, Sanders D W, Bieschke J, Diamond M I. Tau Trimers Are the Minimal Propagation Unit Spontaneously Internalized to Seed Intracellular Aggregation. J Biol Chem. 2015; 290(24):14893-14903.

Nelson P T, Braak H, Markesbery W R. Neuropathology and cognitive impairment in Alzheimer disease: a complex but coherent relationship. J Neuropathol Exp Neurol. 2009; 68(1):1-14.

Neniskyte U, Vilalta A, Brown G C. Tumour necrosis factor alpha-induced neuronal loss is mediated by microglial phagocytosis. FEBS Lett. 2014; 588(17):2952-2956.

Panza F, Lozupone M, Logroscino G, Imbimbo B P. A critical appraisal of amyloid-β-targeting therapies for Alzheimer disease. Nature Reviews Neurology. 2019; 15(2):73-88.

Schlegel R, Dickson R B, Willingham M C, Pastan I H. Amantadine and dansylcadaverine inhibit vesicular stomatitis virus uptake and receptor-mediated endocytosis of alpha 2-macroglobulin. Proc Natl Acad Sci U S A. 1982; 79(7):2291-2295.

Seibenhener M L, Wooten M W. Isolation and culture of hippocampal neurons from prenatal mice. J Vis Exp. 2012(65).

Spangenberg E E, Green K N. Inflammation in Alzheimer's disease: Lessons learned from microglia-depletion models. Brain Behav Immun 2017; 61:1-11.

Sperling R A, Jack C R, Jr., Black S E, et al. Amyloid-related imaging abnormalities in amyloid-modifying therapeutic trials: recommendations from the Alzheimer's Association Research Roundtable Workgroup. Alzheimers Dement. 2011; 7(4):367-385.

Takeuchi H, Iba M, Inoue H, et al. P301S mutant human tau transgenic mice manifest early symptoms of human tauopathies with dementia and altered sensorimotor gating. PLoS One. 2011; 6(6):e21050.

Tse S M, Furuya W, Gold E, et al. Differential role of actin, clathrin, and dynamin in Fc gamma receptor-mediated endocytosis and phagocytosis. J Biol Chem. 2003; 278(5):3331-3338.

Vandenberghe R, Rinne J O, Boada M, et al. Bapineuzumab for mild to moderate Alzheimer's disease in two global, randomized, phase 3 trials. Alzheimers Res Ther. 2016; 8(1):18.

von Kleist L, Stahlschmidt W, Bulut H, et al. Role of the clathrin terminal domain in regulating coated pit dynamics revealed by small molecule inhibition. Cell. 2011; 146(3):471-484.

Wang L H, Rothberg K G, Anderson R G. Mis-assembly of clathrin lattices on endosomes reveals a regulatory switch for coated pit formation. J Cell Biol. 1993; 123(5):1107-1117.

Xu S, Brunden K R, Trojanowski J Q, Lee V M. Characterization of tau fibrillization in vitro. Alzheimers Dement. 2010; 6(2):110-117.

Yamamoto A, Tagawa Y, Yoshimori T, Moriyama Y, Masaki R, Tashiro Y. Bafilomycin A1 prevents maturation of autophagic vacuoles by inhibiting fusion between autophagosomes and lysosomes in rat hepatoma cell line, H-4-II-E cells. Cell Struct Funct. 1998; 23(1):33-42.

Yoshiyama Y, Higuchi M, Zhang B, et al. Synapse loss and microglial activation precede tangles in a P301S tauopathy mouse model. Neuron. 2007; 53(3):337-351.

Yu X, Guo C, Fisher P B, Subjeck J R, Wang X Y. Scavenger Receptors: Emerging Roles in Cancer Biology and Immunology. Adv Cancer Res. 2015; 128:309-364.

Yu L, McPhee C K, Zheng L, et al. Termination of autophagy and reformation of lysosomes regulated by mTOR. Nature. 2010; 465(7300):942-946.

What is claimed is:

1. A chimeric scavenger receptor (CSR) comprising (1) an intracellular signaling domain, wherein the intracellular domain comprises an intracellular domain of a scavenger receptor; (2) a transmembrane domain; and (3) an extracellular domain, wherein the extracellular domain comprises an antigen-specific binding domain.

2. The CSR of claim 1, wherein the transmembrane domain comprises a transmembrane domain of a scavenger receptor.

3. The CSR of claim 1 or 2, wherein the scavenger receptor is FcγRIIb, CD163, CD204, or CD206.

4. The CSR of any one of claims 1-3, wherein the cytoplasmic tail of the intracellular domain is replaced by the cytoplasmic tail of an anti-inflammatory cytokine receptor.

5. The CSR of claim 4, wherein the receptor is an IL-4 receptor, IL-10 receptor, IL-11 receptor, IL-13 receptor, IL-27 receptor, IL-33 receptor, IL-35 receptor, TGF-β receptor, or TSLP receptor.

6. The CSR of any one of claims 1-5, wherein the extracellular domain comprises an extracellular scaffold of a scavenger receptor with the antigen-specific binding domain grafted in place of the scavenger receptor's endogenous binding domain.

7. The CSR of any one of claims 1-6, wherein the antigen-specific binding domain comprises an scFv sequence.

8. The CSR of any one of claims 1-7, wherein the antigen-specific binding domain comprises a single chain monoclonal antibody to any form of the Tau protein.

9. The CSR of claim 8, wherein the single chain monoclonal antibody to o-pTau comprises a variable light chain having a sequence at least 95% identical to
(SEQ ID NO: 6)
DIVMTQTPLSLPVTPGEPASISCRSSQSLVHSHGRTYLHWYLQKPGQSP
QLLIYKVSNRFFGVPDRFSGSGSGTDFTLKISRVEAEDVGVYFCSQTRH
FPYTFGGGTKVEIK.

10. The CSR of claim 8 or 9, wherein the single chain monoclonal antibody to o-pTau comprises a variable heavy chain having a sequence at least 95% identical to
(SEQ ID NO: 7)
EVQLVQSGAEVKKPGSSVKVSCKASGYTFTDYYMNWVRQAPGQGLEWIG
DINPNRGGTTYNQKFKGRVTITVDKSTSTAYMELSSLRSEDTAVYYCAS
YYAVGYWGQGTTVTVSS.

11. The CSR of claim 9 or 10, wherein the variable light chain and variable heavy chain are separated by a linker sequence.

12. The CSR of claim 11, wherein the linker sequence is GGGGSGGGGSGGGGS (SEQ ID NO: 8).

13. The CSR of any one of claims 1-12, further comprising a hinge sequence positioned between the transmembrane domain and the antigen-specific binding domain.

14. The CSR of claim 13, wherein the hinge sequence is VPRDCGCKPCICTV (SEQ ID NO: 9).

15. The CSR of any one of claims 1-14, wherein the chimeric scavenger receptor comprises a sequence at least 95% identical to the sequence of SEQ ID NO: 1 or 5.

16. The CSR of any one of claims 1-14, wherein the chimeric scavenger receptor comprises a sequence at least 95% identical to the sequence of SEQ ID NO: 2 or 4.

17. The CSR of any one of claims 1-14, wherein the chimeric scavenger receptor comprises a sequence at least 95% identical to the sequence of SEQ ID NO: 3.

18. The CSR of any one of claims 1-14, wherein the chimeric scavenger receptor comprises the sequence of SEQ ID NO: 1 or 5.

19. The CSR of any one of claims 1-14, wherein the chimeric scavenger receptor comprises the sequence of SEQ ID NO: 2 or 4.

20. The CSR of any one of claims 1-14, wherein the chimeric scavenger receptor comprises the sequence of SEQ ID NO: 3.

21. A chimeric scavenger receptor (CSR) comprising (1) an intracellular signaling domain, wherein the intracellular domain comprises an intracellular domain of a scavenger receptor; (2) a transmembrane domain; and (3) an extracellular domain, wherein the extracellular domain comprises an antigen-specific binding domain, wherein the antigen-specific binding domain comprises an scFv sequence, wherein the scFv sequence has a variable light chain having a sequence at least 95% identical to
DIVMTQTPLSLPVTPGEPASISCRSSQSLVHSHGRTYLHWYLQKPGQSPQLLIY KVSNRFFGVPDRFSGSGSGTDFTLKISRVEAEDVGVYFCSQTRHFPYTFGGGT KVEIK (SEQ ID NO: 6), and wherein the scFv sequence has a heavy chain having a sequence at least 95% identical to
(SEQ ID NO: 7)
EVQLVQSGAEVKKPGSSVKVSCKASGYTFTDYYMNWVRQAPGQGLEWIG
DINPNRGGTTYNQKFKGRVTITVDKSTSTAYMELSSLRSEDTAVYYCAS
YYAVGYWGQGTTVTVSS.

22. The CSR of claim 21, wherein the transmembrane domain comprises a transmembrane domain of a scavenger receptor.

23. The CSR of claim 21 or 22, wherein the scavenger receptor is FcγRIIb, CD163, CD204, or CD206.

24. The CSR of any one of claims 21-23, wherein the cytoplasmic tail of the intracellular domain is replaced by the cytoplasmic tail of an anti-inflammatory cytokine receptor.

25. The CSR of claim 24, wherein the receptor is an IL-4 receptor, IL-10 receptor, IL-11 receptor, IL-13 receptor, IL-27 receptor, IL-33 receptor, IL-35 receptor, TGF-β receptor, or TSLP receptor.

26. The CSR of claim 21, wherein the variable light chain and variable heavy chain are separated by a linker sequence.

27. The CSR of claim 26, wherein the linker sequence is GGGGSGGGGSGGGGS (SEQ ID NO: 8).

28. The CSR of any one of claims 21-27, further comprising a hinge sequence positioned between the transmembrane domain and the antigen-specific binding domain.

29. The CSR of claim 28, wherein the hinge sequence is VPRDCGCKPCICTV (SEQ ID NO: 9).

30. The CSR of any one of claims 30-29, wherein the chimeric scavenger receptor comprises a sequence at least 95% identical to the sequence of SEQ ID NO: 1, 5, or 11.

31. The CSR of any one of claims 30-29, wherein the chimeric scavenger receptor comprises a sequence at least 95% identical to the sequence of SEQ ID NO: 2 or 4.

32. The CSR of any one of claims 30-29, wherein the chimeric scavenger receptor comprises a sequence at least 95% identical to the sequence of SEQ ID NO: 3.

33. The CSR of any one of claims 30-29, wherein the chimeric scavenger receptor comprises the sequence of SEQ ID NO: 1, 5, or 11.

34. The CSR of any one of claims 30-29, wherein the chimeric scavenger receptor comprises the sequence of SEQ ID NO: 2 or 4.

35. The CSR of any one of claims 30-29, wherein the chimeric scavenger receptor comprises the sequence of SEQ ID NO: 3.

36. A chimeric scavenger receptor (CSR) comprising (1) an intracellular signaling domain, wherein the intracellular domain comprises an intracellular domain of a scavenger receptor, wherein the scavenger receptor is CD163, CD204, or CD206; (2) a transmembrane domain; and (3) an extracellular domain, wherein the extracellular domain comprises an antigen-specific binding domain.

37. The CSR of claim 36, wherein the transmembrane domain comprises a transmembrane domain of the scavenger receptor.

38. The CSR of any claim 36 or 37, wherein the cytoplasmic tail of the intracellular domain is replaced by the cytoplasmic tail of an anti-inflammatory cytokine receptor.

39. The CSR of claim 38, wherein the receptor is an IL-4 receptor, IL-10 receptor, IL-11 receptor, IL-13 receptor, IL-27 receptor, IL-33 receptor, IL-35 receptor, TGF-β receptor, or TSLP receptor.

40. The CSR of any one of claims 36-39, wherein the extracellular domain comprises an extracellular scaffold of a scavenger receptor with the antigen-specific binding domain grafted in place of the scavenger receptor's endogenous binding domain.

41. The CSR of any one of claims 36-40, wherein the antigen-specific binding domain comprises an scFv sequence.

42. The CSR of any one of claims 36-41, wherein the antigen-specific binding domain comprises a single chain monoclonal antibody to any form of the Tau protein.

43. The CSR of claim 42, wherein the single chain monoclonal antibody to o-pTau comprises a variable light chain having a sequence at least 95% identical to
(SEQ ID NO: 6)
DIVMTQTPLSLPVTPGEPASISCRSSQSLVHSHGRTYLHWYLQKPGQSP
QLLIYKVSNRFFGVPDRFSGSGSGTDFTLKISRVEAEDVGVYFCSQTRH
FPYTFGGGTKVEIK.

44. The CSR of claim 42 or 43, wherein the single chain monoclonal antibody to o-pTau comprises a variable heavy chain having a sequence at least 95% identical to
(SEQ ID NO: 7)
EVQLVQSGAEVKKPGSSVKVSCKASGYTFTDYYMNWVRQAPGQGLEWIG
DINPNRGGTTYNQKFKGRVTITVDKSTSTAYMELSSLRSEDTAVYYCAS
YYAVGYWGQGTTVTVSS.

45. The CSR of claim 43 or 44, wherein the variable light chain and variable heavy chain are separated by a linker sequence.

46. The CSR of claim 45, wherein the linker sequence is GGGGSGGGGSGGGGS (SEQ ID NO: 8).

47. The CSR of any one of claims 36-46, further comprising a hinge sequence positioned between the transmembrane domain and the antigen-specific binding domain.

48. The CSR of claim 47, wherein the hinge sequence is VPRDCGCKPCICTV (SEQ ID NO: 9).

49. The CSR of any one of claims 36-48, wherein the chimeric scavenger receptor comprises a sequence at least 95% identical to the sequence of SEQ ID NO: 1, 5, or 11.

50. The CSR of any one of claims 36-48, wherein the chimeric scavenger receptor comprises a sequence at least 95% identical to the sequence of SEQ ID NO: 2 or 4.

51. The CSR of any one of claims 36-48, wherein the chimeric scavenger receptor comprises a sequence at least 95% identical to the sequence of SEQ ID NO: 3.

52. The CSR of any one of claims 36-48, wherein the chimeric scavenger receptor comprises the sequence of SEQ ID NO: 1, 5, or 11.

53. The CSR of any one of claims 36-48, wherein the chimeric scavenger receptor comprises the sequence of SEQ ID NO: 2 or 4.

54. The CSR of any one of claims 36-48, wherein the chimeric scavenger receptor comprises the sequence of SEQ ID NO: 3.

55. A nucleic acid encoding the CSR of any one of claims 1-54.

56. The nucleic acid of claim 55, wherein the sequence encoding the CSR is operatively linked to an expression control sequence.

57. A viral vector comprising the nucleic acid encoding the CSR of any one of claims 1-54.

58. The viral vector of claim 57, wherein the viral vector is a retroviral vector.

59. The viral vector of claim 57, wherein the viral vector is a lentiviral vector.

60. The viral vector of any one of claims 57-59, further comprising a drug selection marker.

61. An engineered immune effector cell comprising the nucleic acid of claim 55 or 56.

62. The engineered cell of claims 61, wherein the immune effector cell is an immune suppressive myeloid cell.

63. The engineered cell of claim 61, wherein the immune effector cell is a monocyte.

64. The engineered cell of any one of claims 61-63, wherein the engineered immune effector cell secretes lower levels of pro-inflammatory cytokines than an equivalent parental immune effector cell.

65. The engineered cell of claim 64, wherein the pro-inflammatory cytokine is TNF-α or IL-6.

66. The engineered cell of any one of claims 61-65, wherein the immune effector cell is a human immune effector cell.

67. A pharmaceutical formulation comprising the engineered cell of any one of claims 61-66 and a pharmaceutically acceptable carrier.

68. A method of treating Alzheimer's disease, frontotemporal dementia, a tauopathy, or a Tau protein-associated impairment in or loss of cognitive function in a patient in need thereof, the method comprising administering an effective amount of a cellular immunotherapy to the patient, wherein the cellular immunotherapy targets a Tau protein.

69. The method of claim 68, wherein the Tau protein is pTau.

70. The method of claim 68, wherein the Tau protein is o-pTau.

71. The method of any one of claims 68-70, wherein the method reduces the levels of Tau, pTau, and/or o-pTau in the brain of the patient.

72. The method of any one of claims 68-70, wherein the method prevents cognitive deterioration in the patient.

73. The method of any one of claims 68-70, wherein the method improves cognitive function in the patient.

74. The method of any one of claims 68-70, wherein the method prevents neural damage in the patient.

75. The method of any one of claims 68-70, wherein the method prevents neuronal fragmentation in the patient.

76. The method of any one of claims 68-70, wherein the cellular immunotherapy is administered to the patient using intraventricular injection.

77. The method of any one of claims 68-70, wherein the cellular immunotherapy is administered to the patient using an intracerebroventricular reservoir.

78. The method of any one of claims 68-70, wherein the cellular immunotherapy is administered by intrathecal injection.

79. The method of any one of claims 68-78, wherein the cellular immunotherapy comprises an engineered cell of any one of claims 61-66.

80. The method of claim 79, wherein the engineered cell is autologous to the patient.

81. The method of any one of claims 68-80, wherein the patient is a human

82. The engineered cells of any one of claims 61-66, for use as a medicament.

83. The engineered cells of any one of claims 61-66, for use in treating Alzheimer's disease, frontotemporal dementia, a tauopathy, or a Tau protein-associated impairment in or loss of cognitive function in a patient.

84. Use of the engineered cells of any one of claims 61-66, in the manufacture of a medicament for treating Alzheimer's disease, frontotemporal dementia, a tauopathy, or a Tau protein-associated impairment in or loss of cognitive function in a patient.


SEQUENCE LISTING (11 sequences)

SEQ ID NO: 1 - protein, 612 aa, Artificial Sequence
  Artificial construct
  MISC_FEATURE (10)..(46): FcyRIIb
  MISC_FEATURE (47)..(158): VL domain
  MISC_FEATURE (159)..(173): linker
  MISC_FEATURE (174)..(288): VH domain
  MISC_FEATURE (289)..(302): hinge
  MISC_FEATURE (303)..(606): FcyRIIb
DIASVDGPTMGILPFLLIPMESNWTVHVFSRTLCHMLLWTAVLNLADIVMTQTPLSLPVTPGEPASISCRSSQSLVHSHGRTYLHWYLQKPGQSPQLLIYKVSNRFFGVPDRFSGSGSGTDFTLKISRVEAEDVGVYFCSQTRHFPYTFGGGTKVEIKGGGGSGGGGSGGGGSEVQLVQSGAEVKKPGSSVKVSCKASGYTFTDYYMNWVRQAPGQGLEWIGDINPNRGGTTYNQKFKGRVTITVDKSTSTAYMELSSLRSEDTAVYYCASYYAVGYWGQGTTVTVSSVPRDCGCKPCICTVAGTHDLPKAVVKLEPPWIQVLKEDTVTLTCEGTHNPGNSSTQWFHNGRSIRSQVQASYTFKATVNDSGEYRCQMEQTRLSDPVDLGVISDALLLQTPQLVFLEGETITLRCHSARNKLLNAISFFHNEKSVRYHAYSSNFSIPKANHSHSGDYYCKGSLGRTLHQSKPVTITVQGTKSSRSLPVLTIVAAVTGIAVAAIVIILVSLVYLKKKQVPALPGNPDHREMGETLPEEVGEYRQPSGGSVPVSPGPPSGLEPTSSSPYNPPDLEEAAKTEAENTITYSLLKHPEALDEETEHDYQNHIDHHHHHH

SEQ ID NO: 2 - protein, 1195 aa, Artificial Sequence
  Artificial construct
  MISC_FEATURE (1)..(38): signal peptide
  MISC_FEATURE (39)..(150): VL domain
  MISC_FEATURE (151)..(165): Linker
  MISC_FEATURE (166)..(280): VH domain
  MISC_FEATURE (281)..(294): Hinge
  MISC_FEATURE (295)..(508): CD163 SRCR 3+4
  MISC_FEATURE (509)..(1066): CD163 cassette
  MISC_FEATURE (1067)..(1195): TM + cytoplasmic domain
MGGHRMVLLGGAGSPGCKRFVHLGFFVVAVSSLLSASADIVMTQTPLSLPVTPGEPASISCRSSQSLVHSHGRTYLHWYLQKPGQSPQLLIYKVSNRFFGVPDRFSGSGSGTDFTLKISRVEAEDVGVYFCSQTRHFPYTFGGGTKVEIKGGGGSGGGGSGGGGSEVQLVQSGAEVKKPGSSVKVSCKASGYTFTDYYMNWVRQAPGQGLEWIGDINPNRGGTTYNQKFKGRVTITVDKSTSTAYMELSSLRSEDTAVYYCASYYAVGYWGQGTTVTVSSVPRDCGCKPCICTVEGADLSLRLVDGVSRCSGRLEVRFQGEWGTVCDDNWDLRDASVVCKQLGCPTAISAIGRVNASEGSGQIWLDNISCEGHEATLWECKHQEWGKHYCHHREDAGVTCSDGADLELRLVGGGSRCAGIVEVEIQKLTGKMCSRGWTLADADVVCRQLGCGSALQTQAKIYSKTGATNTWLFPGSCNGNETTFWQCKNWQWGGLSCDNFEEAKVTCSGHREPRLVGGEIPCSGRVEVKHGDVWGSVCDFDLSLEAASVVCRELQCGTVVSILGGAHFGEGSGQIWGEEFQCSGDESHLSLCSVAPPLDRTCTHSRDVSVVCSRYIDIRLAGGESSCEGRVELKTLGAWGPLCSSHWDMEDAHVLCQQLKCGVAQSIPEGAHFGKGAGQVWSHMFHCTGTEEHIGDCLMTALGAPTCSEGQVASVICSGNQSQTLLPCSSLSPVQTTSSTIPKESEVPCIASGQLRLVGGGGRCAGRVEVYHEGSWGTVCDDNWDMTDANVVCKQLDCGVAINATGSAYFGEGAGAIWLDEVICTGKESHIWQCHSHGWGRHNCRHKEDAGVICSEFMSLRLTNEAHKENCTGRLEVFYNGTWGSIGSSNMSPTTVGVVCRQLGCADNGTVKPIPSDKTPSRPMWVDRVQCPKGVDTLWQCPSSPWKQRQASPSSQESWIICDNKIRLQEGHTDCSGRVEIWHKGSWGTVCDDSWDLNDAKVVCKQLGCGQAVKALKEAAFGPGTGPIWLNEIKCRGNESSLWDCPAKPWSHSDCGHKEDASIQCLPKMTSESHHGTGHPTLTALLVCGAILLVLLIVFLLWTLKRRQIQRLTVSSRGEVLIHQVQYQEMDSKADDLDLLKSSENSNNSYDFNDDGLTSLSKYLPISGIKKGSFRGTLRRKMIIYNPLRLEFKKP

SEQ ID NO: 3 - protein, 612 aa, Artificial Sequence
  Artificial construct
  MISC_FEATURE (1)..(55): cytoplasmic domain
  MISC_FEATURE (56)..(78): transmembrane domain
  MISC_FEATURE (79)..(114): spacer
  MISC_FEATURE (209)..(259): coiled coil
  MISC_FEATURE (277)..(350): collagen-like domain
  MISC_FEATURE (357)..(370): hinge
  MISC_FEATURE (371)..(482): VL domain
  MISC_FEATURE (483)..(497): linker
  MISC_FEATURE (498)..(612): VH domain
MTKEMTENQRLCPHEQEDADCSSESVKFDARSMTASLPHSTKNGPSLQEKLKSFKAALIALYLLVFAVLIPVVGIVTAQLLNWEMKNCLVCSLNTSDTSQGPMEKENTSKVEMRFTIIMEHMKDMEERIESISNSKADLIDTERFQNFSMATDQRLNDILLQLNSLISSVQEHGNSLDAISKSLQSLNMTLLDVQLHTETLNVRVRESTAKQQEDISKLEERVYKVSAEVQSVKEEQAHVEQEVKQEVRVLNNITNDLRLKDWEHSQTLKNITFIQGPPGPQGEKGDRGLTGQTGPPGAPGIRGIPGVKGDRGQIGFPGGRGNPGAPGKPGRSGSPGPKGQKGEKGSVGGSTPLKTVPRDCGCKPCICTVDIVMTQTPLSLPVTPGEPASISCRSSQSLVHSHGRTYLHWYLQKPGQSPQLLIYKVSNRFFGVPDRFSGSGSGTDFTLKISRVEAEDVGVYFCSQTRHFPYTFGGGTKVEIKGGGGSGGGGSGGGGSEVQLVQSGAEVKKPGSSVKVSCKASGYTFTDYYMNWVRQAPGQGLEWIGDINPNRGGTTYNQKFKGRVTITVDKSTSTAYMELSSLRSEDTAVYYCASYYAVGYWGQGTTVTVSS

SEQ ID NO: 4 - protein, 637 aa, Artificial Sequence
  Artificial construct
  MISC_FEATURE (1)..(38): signal peptide
  MISC_FEATURE (39)..(150): VL domain
  MISC_FEATURE (151)..(165): linker
  MISC_FEATURE (166)..(280): VH domain
  MISC_FEATURE (281)..(294): hinge
  MISC_FEATURE (295)..(508): CD163 SRCR 3+4
  MISC_FEATURE (509)..(637): TM + cytoplasmic domain
MGGHRMVLLGGAGSPGCKRFVHLGFFVVAVSSLLSASADIVMTQTPLSLPVTPGEPASISCRSSQSLVHSHGRTYLHWYLQKPGQSPQLLIYKVSNRFFGVPDRFSGSGSGTDFTLKISRVEAEDVGVYFCSQTRHFPYTFGGGTKVEIKGGGGSGGGGSGGGGSEVQLVQSGAEVKKPGSSVKVSCKASGYTFTDYYMNWVRQAPGQGLEWIGDINPNRGGTTYNQKFKGRVTITVDKSTSTAYMELSSLRSEDTAVYYCASYYAVGYWGQGTTVTVSSVPRDCGCKPCICTVEGADLSLRLVDGVSRCSGRLEVRFQGEWGTVCDDNWDLRDASVVCKQLGCPTAISAIGRVNASEGSGQIWLDNISCEGHEATLWECKHQEWGKHYCHHREDAGVTCSDGADLELRLVGGGSRCAGIVEVEIQKLTGKMCSRGWTLADADVVCRQLGCGSALQTQAKIYSKTGATNTWLFPGSCNGNETTFWQCKNWQWGGLSCDNFEEAKVTCSPKMTSESHHGTGHPTLTALLVCGAILLVLLIVFLLWTLKRRQIQRLTVSSRGEVLIHQVQYQEMDSKADDLDLLKSSENSNNSYDFNDDGLTSLSKYLPISGIKKGSFRGTLRRKMIIYNPLRLEFKKP

SEQ ID NO: 5 - protein, 565 aa, Artificial Sequence
  Artificial construct
  MISC_FEATURE (10)..(46): FcyRIIb
  MISC_FEATURE (47)..(158): VL domain
  MISC_FEATURE (159)..(173): Linker
  MISC_FEATURE (174)..(288): VH domain
  MISC_FEATURE (289)..(302): Hinge
  MISC_FEATURE (303)..(559): FcyRIIb
DIASVDGPTMGILPFLLIPMESNWTVHVFSRTLCHMLLWTAVLNLADIVMTQTPLSLPVTPGEPASISCRSSQSLVHSHGRTYLHWYLQKPGQSPQLLIYKVSNRFFGVPDRFSGSGSGTDFTLKISRVEAEDVGVYFCSQTRHFPYTFGGGTKVEIKGGGGSGGGGSGGGGSEVQLVQSGAEVKKPGSSVKVSCKASGYTFTDYYMNWVRQAPGQGLEWIGDINPNRGGTTYNQKFKGRVTITVDKSTSTAYMELSSLRSEDTAVYYCASYYAVGYWGQGTTVTVSSVPRDCGCKPCICTVAGTHDLPKAVVKLEPPWIQVLKEDTVTLTCEGTHNPGNSSTQWFHNGRSIRSQVQASYTFKATVNDSGEYRCQMEQTRLSDPVDLGVISDALLLQTPQLVFLEGETITLRCHSARNKLLNAISFFHNEKSVRYHAYSSNFSIPKANHSHSGDYYCKGSLGRTLHQSKPVTITVQGTKSSRSLPVLTIVAAVTGIAVAAIVIILVSLVYLKKKQVPDNPPDLEEAAKTEAENTITYSLLKHPEALDEETEHDYQNHIDHHHHHH

SEQ ID NO: 6 - protein, 112 aa, Artificial Sequence
  Synthetic sequence
DIVMTQTPLSLPVTPGEPASISCRSSQSLVHSHGRTYLHWYLQKPGQSPQLLIYKVSNRFFGVPDRFSGSGSGTDFTLKISRVEAEDVGVYFCSQTRHFPYTFGGGTKVEIK

SEQ ID NO: 7 - protein, 115 aa, Artificial Sequence
  Synthetic sequence
EVQLVQSGAEVKKPGSSVKVSCKASGYTFTDYYMNWVRQAPGQGLEWIGDINPNRGGTTYNQKFKGRVTITVDKSTSTAYMELSSLRSEDTAVYYCASYYAVGYWGQGTTVTVSS

SEQ ID NO: 8 - protein, 15 aa, Artificial Sequence
  Synthetic sequence
GGGGSGGGGSGGGGS

SEQ ID NO: 9 - protein, 14 aa, Artificial Sequence
  Synthetic sequence
VPRDCGCKPCICTV

SEQ ID NO: 10 - protein, 48 aa, Artificial Sequence
  Synthetic sequence
ALPGNPDHREMGETLPEEVGEYRQPSGGSVPVSPGPPSGLEPTSSSPY

SEQ ID NO: 11 - protein, 612 aa, Artificial Sequence
  Artificial construct
  MISC_FEATURE (10)..(46): FcgammaRIIb
  MISC_FEATURE (47)..(158): VL domain
  MISC_FEATURE (159)..(173): linker
  MISC_FEATURE (174)..(288): VH domain
  MISC_FEATURE (289)..(302): hinge
  MISC_FEATURE (303)..(606): FcgammaR11b
DIASVDGPTMGILPFLLIPMESNWTVHVFSRTLCHMLLWTAVLNLADIVMTQTPLSLPVTPGEPASISCRSSQSLVHSHGRTYLHWYLQKPGQSPQLLIYKVSNRFFGVPDRFSGSGSGTDFTLKISRVEAEDVGVYFCSQTRHFPYTFGGGTKVEIKGGGGSGGGGSGGGGSEVQLVQSGAEVKKPGSSVKVSCKASGYTFTDYYMNWVRQAPGQGLEWIGDINPNRGGTTYNQKFKGRVTITVDKSTSTAYMELSSLRSEDTAVYYCASYYAVGYWGQGTTVTVSSVPRDCGCKPCICTVAGTHDLPKAVVKLEPPWIQVLKEDTVTLTCEGTHNPGNSSTQWFHNGRSIRSQVQASYTFKATVNDSGEYRCQMEQTRLSDPVDLGVISDALLLQTPQLVFLEGETITLRCHSARNKLLNAISFFHNEKSVRYHAYSSNFSIPKANHSHSGDYYCKGSLGRTLHQSKPVTITVQGTKSSRSLPVLTIVAAVTGIAVAAIVIILVSLVYLKKKQVPDNPPDLEEAAKTEAENTITYSLLKHPEALDEETEHDYQNHIALPGNPDHREMGETLPEEVGEYRQPSGGSVPVSPGPPSGLEPTSSSPYHHHHHH